(19) United States
(12) Patent Application Publication
(10) Pub. No.: US 2020/0390854 A1
(43) Pub. Date: Dec. 17, 2020

(54) STIMULATION OF HAIR GROWTH BY SENESCENT CELLS AND SENESCENCE ASSOCIATED SECRETORY PHENOTYPE

(71) Applicant: The Regents of the University of California, Oakland, CA (US)

(72) Inventor: Maksim V. PLIKUS, Irvine, CA (US)

(21) Appl. No.: 16/496,383

(22) PCT Filed: Mar. 21, 2018
(86) PCT No.: PCT/US2018/023624

§ 371 Date: Sep. 20, 2019

Related U.S. Application Data
(60) Provisional application No. 62/475,688, filed on Mar. 23, 2017.

(51) Int. Cl. A61K 38/17; A61B 18/20; A61P 17/14; A61K 8/64; A61K 8/98; A61Q 7/00

(52) U.S. Cl. CPC A61K 38/1754; A61B 18/203; A61P 17/14; A61B 2018/00476; A61K 8/981; A61Q 7/00; A61K 2800/81; A61K 8/64

(57) ABSTRACT

Disclosed herein are methods of treating hair loss affected areas. In some embodiments, the methods of treating hair loss comprise delivering senescent cells or factors derived from senescent cells to the hair loss effected area. Also disclosed herein are methods of making compositions of senescent cells and compositions of senescent cells.

### RELATED APPLICATIONS

This application claims the benefit of U.S. Provisional Application No. 62/475,688, filed Mar. 23, 2017. The content of the aforementioned application is expressly incorporated herein by reference in its entirety.

### BACKGROUND

### Field

Described herein are embodiments of (i) methods of treating hair loss, (ii) methods for obtaining and delivering senescent cells into the skin for the purposes of hair growth stimulation, and (iii) methods for producing and delivering senescent cell associated secretory phenotype (SASP) molecules and their combinations into the skin for the purposes of hair growth stimulation.

### Background

Hair loss (alopecia) results from (i) shortening of the growth phase of the hair regeneration cycle (aka anagen phase) so that progressively shorter hairs are produced and (ii) lengthening the rest phase of the cycle (aka telogen phase) so that hair follicles stop producing new hairs or (iii) the combination of the above two mechanisms.

### SUMMARY

Current available treatments for hair loss involve approaches for extending the duration of the growth phase. There is a deficiency in the art for hair loss treatments that involve shortening the rest phase and for treatments that effectively cause a transition of dormant hair follicles from the telogen to anagen phase.

In some embodiments, a method for enhancing or inducing hair growth in a subject at an area affected by hair loss is provided. In some embodiments, the methods include delivering at least one senescence associated secretory phenotype (SASP) factor, or at least one senescent cell or cell type that secretes said at least one SASP factor, to the subject at the area affected by hair loss. In some embodiments, the at least one senescent cell or cell type comprises at least one cell type that is non-replicative or exhibits a non-replicative phenotype. In some embodiments, the delivery of the at least one senescent cell or cell type or of the at least one SASP factor induces one or more of lengthening an anagen phase and shortening a telogen phase of a hair follicle in the area affected by hair loss. In some embodiments, the lengthening of the anagen phase and/or shortening of the telogen phase of the hair follicle enhances or induces hair growth in the subject at the area affected by hair loss.

In some embodiments, the at least one senescent cell is a melanocyte. In some embodiments, the melanocyte is derived from a nevus skin. In some embodiments, the nevus is a hairy nevus. In some embodiments, the SASP factor is an osteopontin polypeptide. In some embodiments, the osteopontin polypeptide recruits myeloid cells to the area affected by hair loss. In some embodiments, the myeloid cells secrete additional osteopontin polypeptides or other SASP factors to further enhance or induce hair growth in the subject at the area affected by hair loss. In some embodiments, the delivering comprises topical delivery of the at least one senescent cell or cell type. In some embodiments, the topical delivery is performed following application of a microneedle device. In some embodiments, the topical delivery is performed following application of a fractional laser treatment. In some embodiments, the delivering comprises topical delivery of the at least one SASP factor.

In some embodiments, the topical delivery is performed following application of a microneedle device. In some embodiments, the topical delivery is performed following application of a fractional laser treatment.

In some embodiments, a method is provided that comprises delivering at least one type of senescent cell or cell type into a hair loss affected area of the skin.

In some embodiments, a method is provided that comprises injecting at least one factor secreted by senescent cells and that is/are a senescent cell associated secretory phenotype (SASP) molecule into a hair loss affected area of the skin. In some embodiments, a method is provided that comprises exposing at least one type of normal cell to at least one oncogenic factor to induce their senescence.

In some embodiments, a method is provided that comprises exposing at least one normal cell to at least one type of senescence-inducing factor.

In some embodiments, a method is provided that comprises delivering a composition, wherein the composition is made up at least in part, substantially, or completely of factors derived from SASP.

In some embodiments, a method is provided that comprises delivering at least one factor derived from SASP and at least one senescent cell or cell type into a hair loss affected area.

In some embodiments, a method is provided comprising delivering at least one factor derived from SASP and at least one factor derived from an immune cell into a hair loss affected area.

In some embodiments, a method is provided comprising delivering a cocktail of factors produced by co-culturing at least one senescent cell with at least one immune cell or cell type.

In some embodiments, a composition is provided comprising at least one factor derived from SASP.

In some embodiments, the SASP factors include: Angpt14, Ax1, Bmp4, C1qtnf2, C1qtnf5, C1qtnf7, Ccl17, Cc14, Cc15, Cc16, Cc19, Ctsb, Cxc112, Cxc19, Dhh, Dkk3, Fgf7, Frzb, Fstl1, Gdf10, Igfbp2, Igfbp4, Igfbp7, I110, I11a, I11f9, Inhba, Ins16, Mif, Mmp11, Mmp12, Mmp14, Mmp2, Mmp23, Mmp3, Nrg2, Ogn, Omd, Pdgfa, Plat, Postn, Retnia, Sct, Sparc, Spp1, Timp1, Tnf, Tnfaip6, Wif1, and Wisp1. In some embodiments, the SASP factors specific to human nevus skin: ANGPTL7, BAMBI, CCL18, DKKL1, FGFBP2, FRZB, GDF1, GDF10, GDF11, GDF15, IL17D, MMP17, PDGFD, SPP1, TNFSF12, C1QTNT5, NRG3, PLAT, and TIMP2.

In some embodiments, a composition is provided comprising at least one factor derived from senescent cells cultured together with at least one type of immune cell or cell type.

In some embodiments, a composition is provided comprising some or all factors derived from SASP.

in some embodiments, a composition is provided comprising all factors listed in Table 1.1.

The compositions and related methods summarized above and set forth in further detail below describe certain actions taken by a practitioner; however, it should be understood that they can also include the instruction of those actions by another party. Thus, actions such as “transplanting at least one senescent cell type” includes “instructing the transplantation of at least one senescent cell type.”

### BRIEF DESCRIPTION OF THE DRAWINGS

FIG. 1 depicts stages of the oncogene-induced cellular senescence.

FIG. 2 includes images showing large numbers of Trp2-positive senescent melanocytes around a hair follicle in skin of Tyr-Nras^{Q61K} mice, an animal model for hairy nevus (right panel). In normal skin (left panel), Trp2-positive normal melanocytes are present only within the hair follicle (arrow).

FIGS. 3A-3B. The top panels of FIG. 3A depict that the total percentage of the so-called hair follicle bulge stem cells is not significantly altered in the skin of Tyr-Nras^{Q61K} mice. The bottom panels of FIG. 3B show that the percentage of quiescent bulge stem cells is much lower as compared to control mice (0.05% vs. 14.2%) pointing at the hair follicle stem cell over-activation inside of the nevus skin of Tyr-Nras^{Q61K} mice. FIG. 3B shows that according to some embodiments, skin of Tyr-Nras^{Q61K} mice has significantly increased numbers of immune cells (20.7%) as compared to skin from control mice (5.77%).

FIGS. 4A-4G. FIGS. 4A-4B depict congenital melanocytic nevus in a young child. FIGS. 4C-4D include images of experimental and control mice after being shaved at postnatal day 50, and later on postnatal day 62 to show enhanced hair re-growth. FIGS. 4E-F depict histological data and images of experimental and control mice on postnatal day 56. Spots in the LacZ images of bleached skin in FIG. 4F indicate WNT signaling and hair growth. FIG. 4G shows a timeline summary of timing of postnatal sampling performance and data collection.

FIGS. 5A-5E include images and experiments that show oncogene-induced hair growth. FIGS. 5B-2C includes a depiction of an experiment ant resulting data in which cells separated by FACS were injected into wild-type mice. FIG. 5C shows hair growth in response to injected senescent melanocyte cells. FIGS. 5D-5E includes images showing hair growth or a lack of hair growth, in control and experimental mice.

FIGS. 6A-6I and 6H′-6I′. FIGS. 6A-6G depict data and information generated through a bioinformatics analysis of gene expression. The plot in FIG. 6A shows the results of a principal component analysis (PCA) indicating that the gene expression varies between stem cells of wild-type and experimental mice. FIG. 6B includes a heat map of differentially expressed genes between wild-type and experimental mice. FIG. 6B also includes a Venn diagram of genes upregulated or downregulated in hairy nevus stem cells from experimental mice compared to wild-type mice at postnatal days 30 and 56. FIG. 6C depicts a gene ontology analysis of the heat map data from FIG. 6D depicts single cell RNA-seq data for experimental and control cells. FIG. 6E depicts violin plots from a single-cell sequencing analysis. FIG. 6F includes plots of qRT-PCR data that validate RNA-sequencing data for selected genes. FIG. 6G is a table of signaling-transcription-related genes that are downregulated (left rectangles) or upregulated (right rectangles) in experimental mice compared to wild-type. FIGS. 6H and 6H′ depict data and histology images from pulse-chase experiments in hair follicle stem cells. FIG. 6I includes images of cultured GFP labeled bulge stem cells and hair germ (HG) cells. FIG. 6I′ includes graphical depictions of average number of attached cells or passages until quiescence of GFP-labeled bulge stem cells and HG cells.

FIGS. 7A-7N include images and graphical data of analyses of melanocytes. FIGS. 7A-7D, 7G and 7H include an analysis of bulk RNA sequencing. FIG. 7D includes a Venn diagram that includes signaling factors increased in mutant melanocytes extracted from skin of mice with nevi (top circle), and SASP factors identified in cultured cells (left and right circles). FIGS. 7E and 7F include plots of data from single-cell RNA sequencing. FIG. 7I shows plots of measured secreted and surface-bound osteopontin (Spp1) protein in skin cells. FIG. 7J depicts western blot data of multiple isoforms of osteopontin in mouse nevus skin. FIG. 7K includes images of a genetic reporter assay for Spp1. FIG. 7L depicts data and a three-colored timeline of time-points where data were collected. The data indicate that Spp1 is not the only molecule involved in nevus hair growth. FIG. 7M depicts photographs and graphical data of wound-induced hair growth in control and experimental mice. FIG. 7N depicts photographs and graphical data of hair growth after Spp1 injection or a bovine serum albumin (BSA) control.

FIGS. 8A-8K and 8H′-8K′. FIGS. 8A-8D include RNA sequencing data of myeloid cells. FIG. 8A is a PCA plot. FIG. 8B is a heat map. FIG. 8C depicts a gene ontology analysis. FIG. 8D includes a Venn diagram and summary of differentially expressed genes in mutant myeloid cells identified by RNA sequencing analysis. The Venn diagram includes SASP factors identified in cultured cells (left circle), and SASP factors of melanocytes (bottom circle) and myeloid cells (right circle). FIG. 8E includes plots of qRT-PCR data validating gene expression data for the RNA sequencing analysis shown in FIGS. 8A-8D. FIGS. 8F and 8G include single cell gene expression analyses of myeloid cells. FIGS. 8H and 8I includes images of myeloid cells in skin of wild-type and experimental mice. FIGS. 8H′ and 8I′ are plots that include summary quantification information of numbers of myeloid cells in wild-type and experimental mouse skin. FIG. 8J includes images of mouse skin injected with BSA or Spp1, and shows an influx of immune cells in response to the Spp1. FIGS. 8K and 8K′ depict images and graphical data confirming involvement of myeloid cells in sustaining hair growth in nevus skin.

FIGS. 9A-9G and 9D′-9G′. FIG. 9A depicts plots of cd44 expression in various cell types. FIG. 9B shows isoforms of cd44 arranged by cell type. FIG. 9C includes images of a genetic reporter assay for cd44. FIGS. 9D and 9D′ include images and graphical data depicting skin injected with beads containing osteopontin. FIGS. 9E and 9E′ include images and graphical data depicting wound-induced hair growth. The data in these figures indicate that osteopontin acts on cd44 to produce its results. FIGS. 9F and 9F′ include images of experimental mice at postnatal day 52 in telogen and anagen. FIG. 9G includes a summary of time points in which data were collected for experimental mice. FIG. 9G′ includes plots quantifying ectopic anagen HFs in experimental mice at the time points indicated in FIG. 9G.

FIGS. 10A-10I and 10G′-10I′ include depictions of data from three human subjects with facial hairy nevi. RNA sequencing data were obtained for hairy nevus skin of each patient, as well as control non-nevus skin from each patient. FIG. 10A is a PCA analysis of the RNA sequencing data. FIG. 10B is a heat map of the RNA sequencing data. FIG. 10C is a gene ontology analysis of the RNA sequencing data. FIG. 10D shows genes that were upregulated or downregulated in the human nevus skin compared to the human control skin. FIG. 10E includes a summary of molecules upregulated in human nevus skin compared to control skin, and a Venn diagram showing overlaps between the genes upregulated in human nevus skin (top-left circle), genes upregulated in experimental mice (bottom circle), and in cultured cells (top-right circle). FIG. 10F includes a graphical depiction showing expression of genes in human nevi as indicated by qRT-PCR. FIGS. 10G-10I, and 10G′-10F include histology images of human nevus and control skin.

FIGS. 11A-11F depict histological data and images of experimental and control mice on postnatal days 15, 23, 44, 62, 69 and 100, and indicate that at each of the time points the experimental mice are growing hair.

FIGS. 12A-12B depict histological data and images of control and experimental Tyr-Nras^{Q61K} mice on postnatal days 56 and 100 after being crossed with an albino background or not. These data indicate that melanin is not necessarily the cause of hair growth seen in Tyr-Nras^{Q61K} mice.

FIGS. 13A-13B show that ectopic hair growth can be induced when fluorescent senescent melanocytes isolated by cell sorting from the skin of Tyr-CreER^{T2};tdTomato mice (panels in FIG. 13A) are injected into the skin of immune-compromised SCID mice (see drawing in FIG. 13A, and panels in FIG. 13B).

FIGS. 14A-14B include images of hair growth or a lack of hair growth, in control and experimental mice.

FIG. 15 depicts an analysis of cell cycle states in single cell sequencing data.

FIG. 16 depicts violin plots from a single-cell sequencing analysis.

FIG. 17 depicts violin plots from a single-cell sequencing analysis.

FIGS. 18A-18D depict an RNA sequencing analysis of HG cells.

FIGS. 19A-19E depict an RNA sequencing analysis and qRT-PCR validation of the RNA sequencing data, of DP fibroblasts.

FIGS. 20A-20D depict an RNA sequencing analysis of cd45 hematopoietic

FIGS. 21A-21C and 21B′-21C′ include graphical data of a detailed analysis of an RNA-sequencing analysis.

FIG. 22 is a pictures of hair follicles in telogen (arrested phase) ,nd anagen (growth phase). The figure indicates where bulge, HG and DP cells reside in relation to each other within a hair follicle. The figure depicts a summary of signaling changes in each cell type in nevus skin.

FIGS. 23A-23B includes images of a genetic reporter assay for Spp1. Expression of Spp1 is shown at 2 time points (whole mount and detailed sections). These data indicate that Spp1 is produced more in mutant mice than wild-type.

FIGS. 24A-24F include images of experimental and control mouse skin at various postnatal time points.

FIGS. 25A-25B includes images showing a role of cd44 in hair growth.

FIGS. 26A-26D include images of experimental mice at postnatal days 30, 56, 69 and 100 in telogen and anagen.

FIGS. 27A-27D include images of experimental and control mice and skin at various postnatal time points.

### DETAILED DESCRIPTION

Hair loss (aka alopecia) in humans results from two changes in the so-called hair growth cycle, the physiological cyclic process of hair synthesis by the hair follicle: (a) shortening of the growth phase (aka anagen), so that progressively shorter and shorter hairs are being produced; and (b) lengthening of the rest phase (aka telogen), so that hair follicles stop making new hairs all together for a prolonged period of time.

Pharmacological solutions to hair loss involve modulating signaling pathways that normally induce longer growth phase and shorter rest phase with either systemically, locally or topically delivered pharmaceuticals. To-date, the most prominent anti-hair loss effect was recorded for the agents that in one way or another reduce androgen signaling, and their effect is primarily directed toward lengthening the growth phase. The effect of reduced androgen signaling on telogen-to-anagen transition is not significant. For this reason, the anti-hair loss effect of Finasteride, for example, is very gradual and takes several years to fully show. Specifically, dormant hair follicles need to spontaneously enter a new anagen phase first for the anagen phase lengthening effect of Finasteride to be observable.

In mice, many other signaling pathways have been identified whose activation or suppression can promote transition of hair follicles from telogen to new anagen phase. However, for the most part, their effects on growth phase activation in humans have not been studied. Furthermore, some of the key signaling molecules that can stimulate new growth phase are also potent growth factors that have many other, often undesirable off-target side effects. For example, WNT signaling, which can active hair growth in mice, can also signal to promote growth of cancer cells. Therefore, use of WNT molecules for treating hair loss might result in higher risk of skin tumorigenesis,

To-date, no therapeutic solution exists in humans for (i) efficiently activating new hair growth phase, and (ii) simultaneously increasing duration of the growth phase, and thus length of hairs.

### “Hairy Nevus”

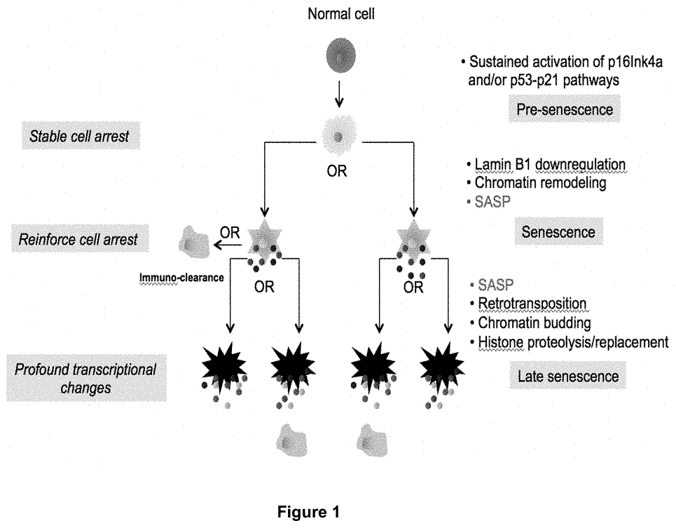

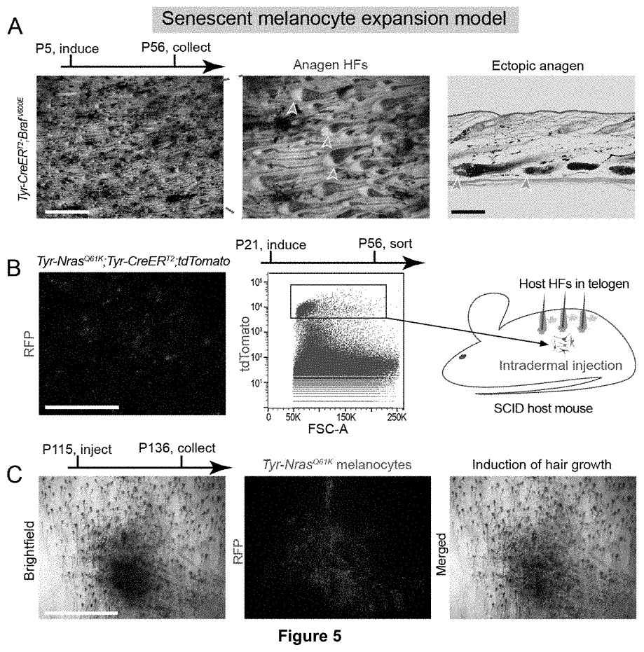

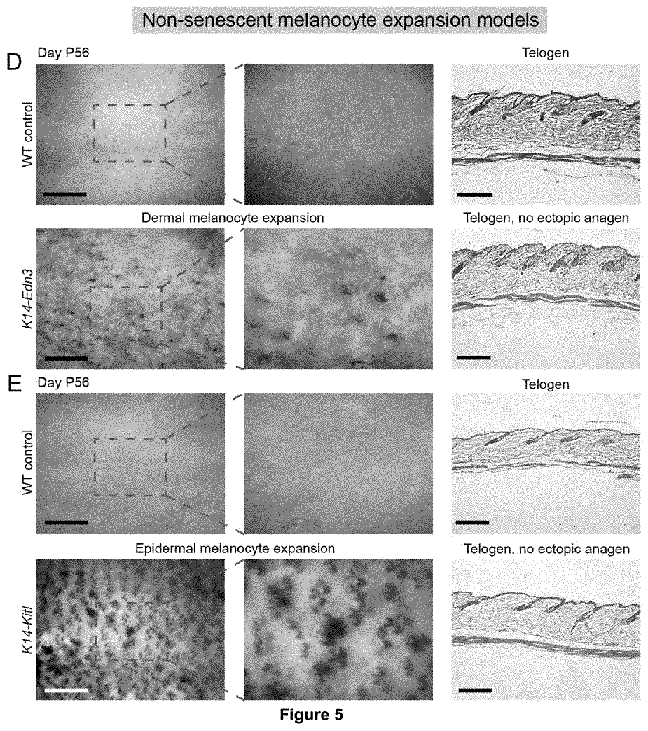

“Hairy nevus” is an under-studied and very poorly understood phenomenon. Nevus is a type of benign skin lesion that is pigmented and contains increased number of specialized melanocytes. Unlike normal skin, hairy nevus skin lesions contain many so-called senescent melanocytes that become senescent as the result of acquiring an oncogenic mutation. An example of human hair nevus with enhanced hair growth as compared to surrounding non-nevus skin is shown in FIG. 5A. Typical stages of oncogene-induced senescent cell formation, including activation of the so-called Senescence Associated Secretory Phenotype (SASP) are shown in FIG. 1.

Normal body hairs, called vellus hairs, are typically very short, thin and often non-pigmented and, thus, barely visible. However, these hairs transform into prominent scalp-like hairs that are long, thick and pigmented (aka terminal hairs) once inside of the nevus boundaries. Clinically, vellus-to-terminal hair transformation is highly desirable and forms basis for treating hair loss, when achieving many terminal hairs is the ultimate goal.

Studies using several mouse models tested the hypothesis of whether specialized senescent melanocytes in the nevus skin can drive activation of hair growth. These studies showed that senescent melanocytes indeed prominently enhance hair growth. The studies also showed that senescent melanocytes achieve this effect via SASP factors that they secrete. Generally, SASP represents a set of secreted signaling molecules, enriched in members of inflammatory signaling pathways that are produced by all types of senescent cells, including senescent melanocytes.

SASP Profile in Senescent Melanocytes Derived from “Hairy Nevus”

The SASP profile of senescent melanocytes derived from hairy nevus skin was evaluated and established by RNA-sequencing on sorted cells. From this analysis, multiple candidate molecular players have been identified that appear to be responsible for promoting hair growth in the nevus, either as individual molecules, or in combination. Taken together, based on this data it was determined that senescent-cell derived SASP factors are the primary drivers of enhanced hair growth. This indicates that exposing dormant (telogen) hair follicles to either senescent cells or senescent cell-derived SASP or components of SASP, as in accordance with several embodiments disclosed herein, induces their activation and enhance hair growth.

Moreover, the data shows that senescent melanocyte-produced SASP also induces recruitment into the skin and activation of immune cells, specifically macrophages. RNA-sequencing studies on sorted nevus skin macrophages showed that they also secrete many of the same SASP factors and other additional inflammatory cytokines. Thus, in effect macrophages and their secreted molecules amplify and potentiate hair growth-inducing effect of senescent cell-derived SASP factors. Thus, SASP or components of SASP with macrophage-derived signaling factors may result in potentiation of the hair growth inducing effect.

### Embodiments of Utilizing Senescent Cells, SASP and Immune Cell-Derived Factors for Inducing Hair Growth

In some embodiments described herein, SASP factors are collected and purified for skin injection from cultured senescent melanocytes or any other type of senescent cell (fibroblasts, keratinocytes, etc.). Advantageously, it has been determined that different senescent cells can be used because different varieties of senescent cells share large portions of their SASP molecular profiles. In some embodiments described herein, secreted factors are collected and purified from the co-culture of senescent cells with the immune cells, such as macrophages. Once collected, these “bioactive factor cocktails” can be delivered into skin via a number of ways, including but not limited to direct intra-dermal injection, topical delivery following application of a micro-needle device or fractional laser treatment.

In some embodiments described herein hair growth is stimulated by (i) senescent cells or (ii) senescent cell derived bioactive SASP cocktail of signaling molecules, or (iii) signaling molecule cocktails produced by a combination of senescent cells and macrophages.

### Embodiments of Methods of Treatment of Hair Loss Affected Areas

In some embodiments, a method is provided that comprises transplanting at least one senescent cell type into a hair loss affected area. In some embodiments, a method is provided that comprises transplanting a population of senescent cells into a hair loss affected area.

In some embodiments, the population of senescent cells is a pure population of senescent cells. For example, the method comprises transplanting a population of senescent cells that are greater than 70% pure, greater than 80% pure, greater than 90% pure, or greater than 95% pure.

In some embodiments, a method is provided that comprises delivering at least one senescent cell and at least one factor derived from SASP to a hair loss affected area.

Any of the senescent cells described herein can be derived from any organism. In some embodiments, the senescent cells are human senescent cells. In some embodiments, the senescent cells are any one or more of senescent melanocytes, senescent fibroblasts, senescent keratinocytes, or senescent adipocytes. In some embodiments, the senescent cell is any cell type that is senescent or has entered a senescent phenotype. In some embodiments, a senescent phenotype includes a non-replicative phenotype.

In some embodiments, a method is provided that comprises transplanting at least one factor derived from SASP into a hair loss affected area. In some embodiments, a method is provided that comprises adding at least one factor produced by immune cells into a hair loss affected area. In some embodiments, a method is provided that comprises adding at least one factor from SASP and at least one factor from immune cells into a hair loss affected area.

In some embodiments, a method is provided that comprises delivering a composition into a hair loss affected area, wherein the composition is made up at least in part, substantially or completely of factors derived from SASP. In some embodiments, a method is provided that comprises delivering at least one factor derived from SASP and at least one factor derived from at least one immune cell into a hair loss affected area.

In some embodiments, a method is provided comprising delivering at least one factor derived from culturing senescent cells with at least one type of immune cell into a hair loss affected area. The senescent cells are any senescent cells in the skin. In some embodiments, the at least one senescent cell comprises any one or more of senescent melanocytes, senescent fibroblasts, senescent keratinocytes, or senescent adipocytes. The immune cells are any immune cells. In some embodiments, the immune comprises any one or more of neutrophils, eosinophils, basophils, lymphocytes, monocytes, and macrophages.

In some embodiments, a method is provided comprising delivering a cocktail of factors produced by co-culturing at least one senescent cell with at least one immune cell. The at least one senescent cell is any senescent in cell found in the skin. In some embodiments, the at least one senescent cell is any one or more of senescent melanocytes, senescent fibroblasts, senescent keratinocytes, or senescent adipocytes. The at least one immune cell is any immune cell type. In some embodiments, the at least one immune cell is any one or more of neutrophils, eosinophils, basophils, lymphocytes and monocytes. In some embodiments, the immune cell is a macrophage.

In some embodiments, a method is provided comprising delivering at least one senescent cell and at least one factor derived from senescent cells into a hair loss affected area. The at least one senescent cell is any senescent cell. In some embodiments, the senescent cell is any one or more of senescent melanocytes, senescent fibroblasts, senescent keratinocytes, and senescent adipocytes.

in some embodiments, in any of the methods described herein that comprise delivering one or more factors derived from SASP, including but not limited to any one or more of the factors listed in Table 1.

TABLE 1
Gene name

Angiogenin (ANG)
Amphiregulin (AREG)
C-C motif chemokine ligand 1 (CCL1)
C-C motif chemokine ligand 11 (CCL11)
C-C motif chemokine ligand 13 (CCL13)
C-C motif chemokine ligand 16 (CCL16)
C-C motif chemokine ligand 2 (CCL2)
C-C motif chemokine ligand 20 (CCL20)
C-C motif chemokine ligand 25 (CCL25)
C-C motif chemokine ligand 26 (CCL26)
C-C motif chemokine ligand 3 (CCL3)
C-C motif chemokine ligand 7 (CCL7)
C-C motif chemokine ligand 8 (CCL8)
Colony stimulating factor 2 (CSF2)
Colony stimulating factor 3 (CSF3)
Cathepsin B (CTSB)
C-X-C motif chemokine ligand 1 (CXCL1)
C-X-C motif chemokine ligand 11 (CXCL11)
C-X-C motif chemokine ligand 12 (CXCL12)
C-X-C motif chemokine ligand 13 (CXCL13)
C-X-C motif chemokine ligand 2 (CXCL2)
C-X-C motif chemokine ligand 3 (CXCL3)
C-X-C motif chemokine ligand 8 (CXCL8)
Dickkopf WNT signaling pathway inhibitor 1 (DKK1)
Epidermal growth factor (EGF)
Epidermal growth factor receptor (EGFR)
Epiregulin (EREG)
Fibroblast growth factor 2 (FGF2)
Fibroblast growth factor 7 (FGF7)
Fibronectin 1 (FN1)
Heparin binding EGF like growth factor (HBEGF)
Hepatocyte growth factor (HGF)
Intercellular adhesion molecule 1 (ICAM1)
Intercellular adhesion molecule 3 (ICAM3)
Interferon gamma (IFNG)
Insulin like growth factor 1 (IGF1)
Insulin like growth factor binding protein 1 (IGFBP1)
Insulin like growth factor binding protein 2 (IGFBP2)
Insulin like growth factor binding protein 3 (IGFBP3)
Insulin like growth factor binding protein 4 (IGFBP4)
Insulin like growth factor binding protein 5 (IGFBP5)
Insulin like growth factor binding protein 6 (IGFBP6)
Insulin like growth factor binding protein 7 (IGFBP7)
Interleukin 12B (IL12B)
Interleukin 13 (IL13)
Interleukin 15 (IL15)
Interleukin 1 alpha (IL1A)
Interleukin 1 beta (IL1B)
Interleukin 21 (IL21)
Interleukin 4 (IL4)
Interleukin 6 (IL6)
Interleukin 6 signal transducer (IL6ST)
Interleukin 7 (IL7)
Galectin 9 (LGALS9)
Macrophage migration inhibitory factor
(glycosylation-inhibiting factor) (MIF)
Major intrinsic protein of lens fiber (MIP)
Nerve growth factor (NGF)
Platelet derived growth factor subunit B (PDGFB)
Phosphatidylinositol glycan anchor biosynthesis
class F (PIGF)
Plasminogen activator, urokinase receptor (PLAUR)
Prolactin (PRL)
Serpin family B member 2 (SERPINB2)
Serpin family E member 1 (SERPINE1)
Osteopontin (SPP1)
Transforming growth factor beta 1 (TGFB1)
Transforming growth factor beta 2 (TGFB2)
Tumor necrosis factor (TNF)
TNF receptor superfamily member 10c (TNFRSF10C)
TNF receptor superfamily member 11b (TNFRSF11B)
TNF receptor superfamily member 1A (TNFRSF1A)
TNF receptor superfamily member 1B (TNFRSF1B)
Tumor necrosis factor superfamily member 13 (TNFSF13)
Thymic stromal lymphopoietin (TSLP)
Wnt family member 16 (WNT 16)
Wnt family member 5A (WNT5A)

In some embodiments, one or more SASP factors are produced by one or more cells. In some embodiments, the one or more cells include at least one of a mammalian cell, a human cell, a mouse cell, a rat cell, a bacterial cell, a yeast cell, and/or any other type of cell capable of producing the one or more SASP factors. In some embodiments, one or more SASP factors are secreted from a cell. In some embodiments, the one or more SASP factors are secreted into a medium. In some embodiments, one or more SASP factors are isolated and/or purified after being secreted. For example, the one or more SASP factors may be isolated and/or purified from a supernatant after centrifuging cells and media associated with the cells. In some embodiments, one or more SASP factors are isolated and/or purified without being secreted from cells. In some embodiments, one or more SASP factors are produced recombinantly in a cell, such as through the use of standard molecular biology techniques. In some embodiments, one or more SASP factors are produced synthetically. In some embodiments, one or more SASP factors are purchased commercially.

In some embodiments, the SASP factors include mouse SASP factors such as Angpt14, Ax1, Bmp4, C1qtnf2, C1qtnf5, C1qtnf7, Ccl17, Cc14, Cc15, Cc16, Ccl9, Ctsb, Cxcl12, Cxcl9, Dh, Dkk3, Fgf7, Frzb, Fstl1, Gdf10, Igfbp2, Igfbp4Igfbp7, I110, I11, Il1f9, Inhba, Ins16, Mif, Mmp11, Mmp12, Mmp14, Mmp2, Mmp23, Mmp3, Nrg2, Ogn, Omd, Pdgfa, Plat, Postn, Retnla, Set, Sparc, Spp1, Timp1, Tnf, Tnfaip6, Wif1, and/or Wisp1. In some embodiments, the SASP factors include human nevus skin SASP factors such as ANGPTL7, BAMBI, CCL18, DKKL1, FGFBP2, FRZB, GDF1, GDF10, GDF11, GDF15, IL17D, MMP17, PDGFD, SPP1, TNESF12, C1QTNF5, NRG3, PLAT, and/or TIMP2. Some embodiments, include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, any number therebetween, or more, of the SASP factors described or identified herein.

In any of the methods described herein that comprise delivering at least one or more factors derived from immune cells, the factors are derived from any white blood cell type. In some embodiments, the factors derived from any white blood cell type are produced from hematopoietic stem cells. In some embodiments, the factors derived from white blood cells are derived from any one or more of neutrophils, eosinophils, basophils, lymphocytes and monocytes. In some embodiments, the immune cell from which the one or more factors derive is a macrophage.

In some embodiments, delivering comprises at least one intradermal injection. In some embodiments, delivering comprises multiple repetitive intradermal injections. In some embodiments, the delivering comprises topical delivery. In some embodiments, the topical delivery follows application of a microneedle device or a fractional laser treatment.

Any of the compositions disclosed herein can he delivered into a hair loss affected area through any of the methods disclosed herein.

### Embodiments of Methods of Producing Senescent Cells

In some embodiments, a method of producing senescent cells is provided that comprises exposing one or more normal cells to one or more oncogenic factors. In some embodiments, a method of producing senescent cells is provided that comprises exposing one or more normal cells to one or more senescent inducing factors. In some embodiments, a method of producing senescent cells is provided that involves repetitive passaging of cells to achieve replicative senescence.

### Embodiments of Compositions

In some embodiments, a composition is provided that comprises at least one factor derived from SASP. In some embodiments, a composition is provided that comprises at least one factor derived from SASP and at least one factor derived from one immune cell type. In some embodiments, at least one factor derived from SASP is any one or more of the factors listed in Table 1.

In compositions that comprise factors derived from immune cells, the factors are derived from any one or more white blood cells or any combination of white blood cells. In some embodiments, the factors derived from any one or more of white blood cells that are produced from hematopoietic stem cells. In some embodiments, the factors from white blood cells are derived from any one or more of neutrophils, eosinophils, basophils, lymphocytes and monocytes. In some embodiments, the immune cells from which the one or more factors derive, are macrophages.

In some embodiments, a composition is provided that comprises at least one factor derived from senescent cells that are cultured with at least one type of immune cell. The senescent cells can comprise any senescent cell found in the skin. In some embodiments, the senescent cells comprise any one or more of the following: senescent melanocytes, senescent fibroblasts, senescent keratinocytes, and senescent adipocytes. In some embodiments, the at least one factor derived from senescent cells include but are not limited any one or more of the factors listed in Table 1.1.

In some embodiments, a composition is provided that comprises all factors derived from SASP. In some embodiments, the composition is provided comprising each of the factors listed in Table 1.1.

In some embodiments, a composition is provided that includes one or more SASP factors. In some embodiments, the composition includes a medium or supernatant containing one or more SASP factors. In some embodiments, the SASP factors that are included in the medium or supernatant, are secreted by a cell into the medium or supernatant.

### EXAMPLES

Some aspects of the embodiments discussed above are disclosed in further detail in the following examples, which are not in any way intended to limit the scope of the present disclosure

### Example 1

### Verify that the Transuenic Mouse Lines Tvr-Nras^{Q61K} and Tyr-CreER^{T2}, Braf^{V600E} can be used as Models for Overactive Hair Growth

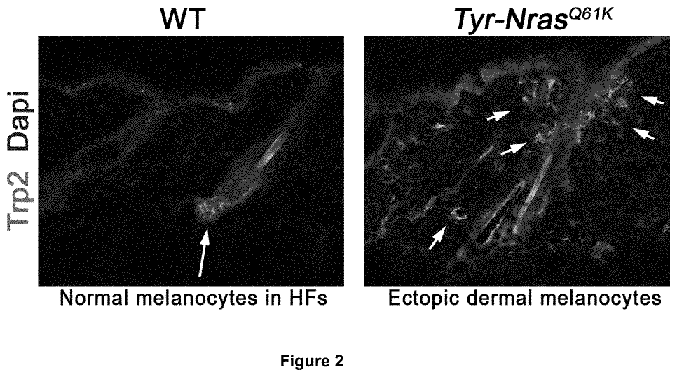

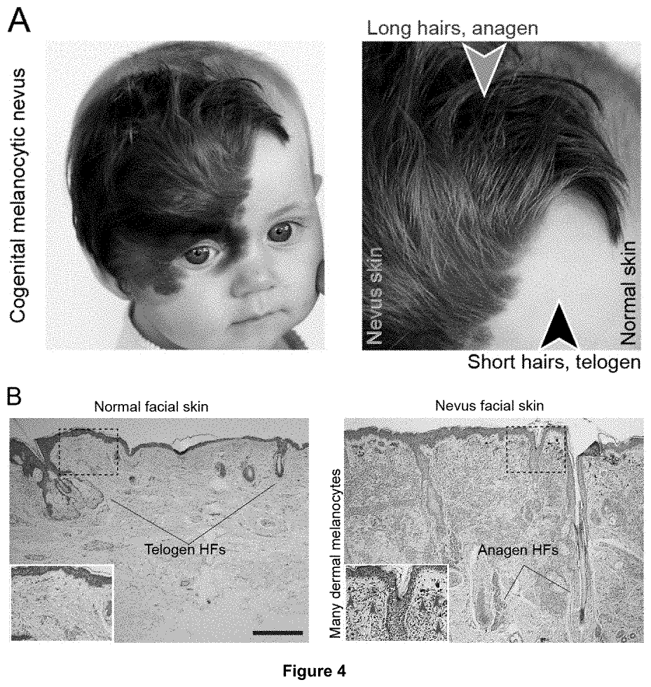

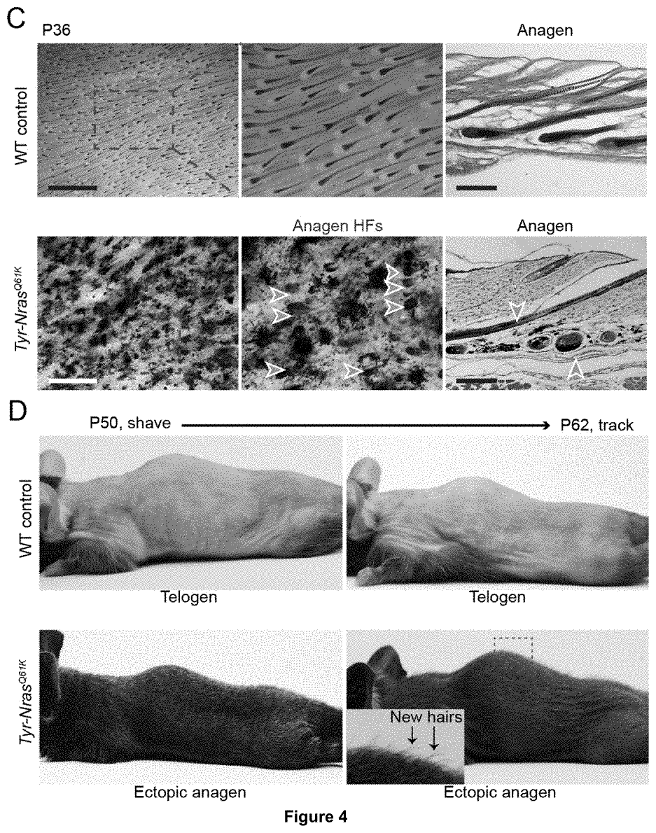

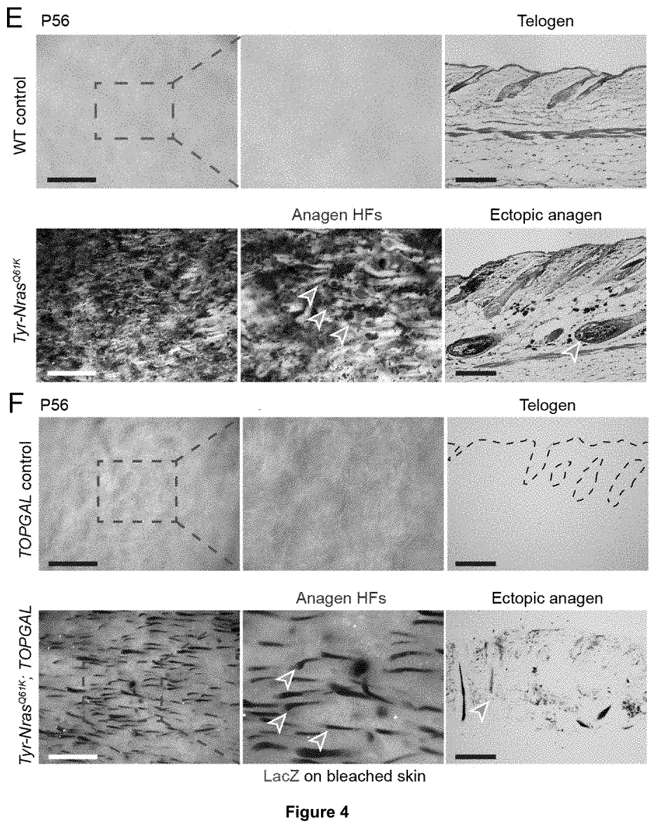

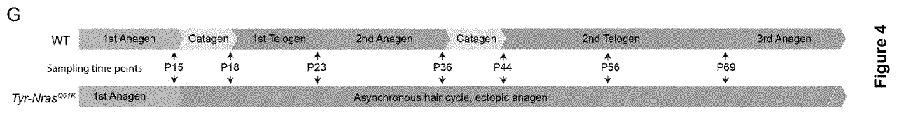

Several transgenic mice were used as models for the oncogene-induced senescence in melanocyte cell lineage to verify that they display overactive hair growth, replicating human hairy nevus lesions. One such mouse model is Tvr-Nras^{Q61K}. Similar to human hairy nevus, skin of these mice showed large number of ectopic senescent melanocytes (FIG. 2). Hair growth in the skin of Tyr-Nras^{Q61K} mice was dramatically enhanced at all time points examined. While normal, control mice typically show lack of active hair growth, Tyr-Nras^{Q61K} mice displayed many growing hair follicles, as shown in FIG. 4F (bottom panels). The Tyr-CreER^{T2}; Braf^{V600E} mice also show enhanced hair growth (FIG. 5A) indicating that it was a model for senescent nevus.

### Example 2

### Using the Pulse-Chase Technique to Show that Hair Follicle Stem Cells are Active in the Skin of Tyr-Nras^{Q61K} Mice

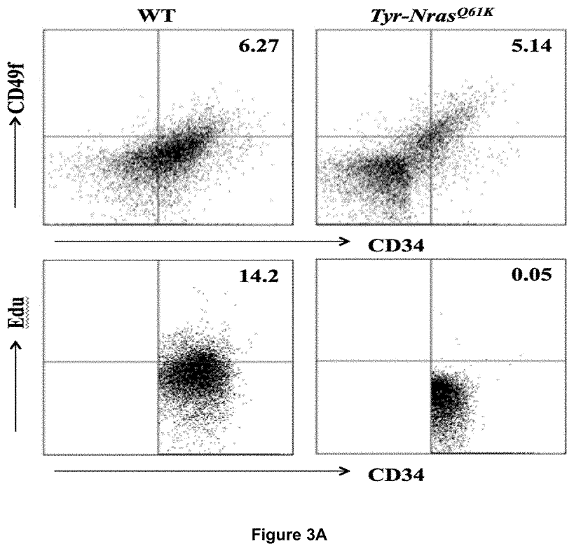

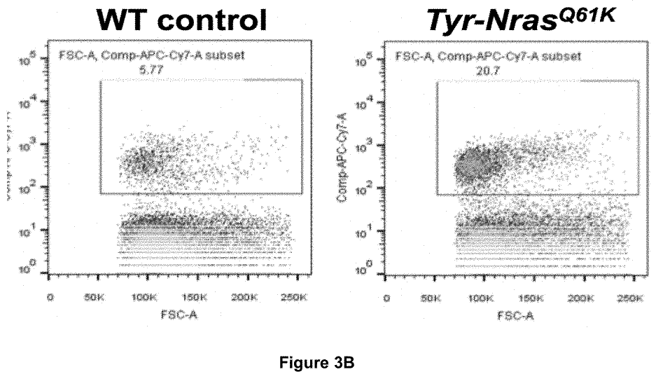

The so-called pulse-chase technique showed that hair follicle stem cells (aka bulge stem cells) are less quiescent and more active in the skin of Tyr-Nras^{Q61K} mice as compared to control mice (FIG. 3). This data supports that presence of senescent cells results in enhanced hair growth by, for example, activating hair follicle stem cells.

### Example 3

### Verifying that Ectopic Hair Growth in Skin is the Result of Senescent Melanocytes

Injection of fluorescently labeled senescent melanocytes in the skin of mice resulted in ectopic activation of hair growth (FIGS. 5B and 5C). To verify that the hair growth was the result of senescent melanocytes, rather than other types of melanocytes, hair growth was examined in two mouse models for expanded normal, non-senescent melanocytes, the K14-Edn3 mice and the K14-Kit1 mice. In both of these mice, despite expanded melanocytes, no enhancement of hair growth was observed (FIGS. 5A).

### Example 4

### Increased Numbers of Hematopoietic Cells are Observed in the Nevus Skin of Tyr-Nras^{Q61K} Mice

In certain embodiments as shown by FACS analysis in FIG. 3B, the nevus skin of Tyr-Nras^{Q61K} mice had increased numbers of hematopoietic cells. This may correspond with increased numbers of immune cells playing a role in SASP signaling.

### Example 5

### Signaling by Senescent Cells Hyper-Activates Skin Stem Cell Niche

The present example shows that hair follicle stem cells can exist in quiescence, and change their transcriptome and composition, and that hair regeneration dramatically enhances in the presence of senescent melanocytes. It was shown here that the latter activate a senescence-associated secretory phenotype (SASP), containing pro-inflammatory factors. Osteopontin is a new SASP factor involved in hair regeneration. Osteopontin injection was shown to be sufficient to induce new hair growth, and to recruit myeloid cells which amplify osteopontin levels and enhance SASP effect on hair regeneration. Deletion of osteopontin, its receptor, Cd44, or depletion of myeloid cells all markedly reversed enhanced hair regeneration by senescent melanocytes. While conventionally senescent cells are viewed as being detrimental for tissue regeneration potential, it is here shown that they can enhance regeneration by enriching stem cell niche for SASP signaling and immune cell modulation.

It was shown here that senescent cells can induce dramatic loss of quiescence by tissue stem cells (SCs) and this is enabled by their unique secretome, the SASP. It is here shown that senescent melanocytes in pigmented nevus skin (aka mole) signal via SASP to hyper-activate hair SCs, leading to prominently enhanced hair regeneration. SASP recruits myeloid cells which, in turn, amplify and enrich it for novel pro-regenerative signaling factors. Osteopontin is herein identified as a novel SASP factor, responsible for enhanced hair regeneration. Activation of tissue progenitors by aged tissue cells provides a novel paradigm in SC biology.

### Hair Regeneration is Hyper-Activated in Nevus Skin Containing Senescence Melanocytes

Cyclic hair regeneration is tightly controlled at the level of stem cell quiescence (Yi, 2017), and naturally occurring conditions of excessive hair growth are rare. A hairy pigmented nevus is a type of benign skin lesion in humans with prominently enhanced hair growth (FIGS. 4A and 4B). The mechanism of excessive hair growth in nevi is not understood. Nevi form as the result of an oncogene mutation, commonly in Nras or Braf, in skin melanocytes (Roh et al,, 201:5). This activates oncogene-induced senescence (OIS) in affected cells (Dhomen et al., 2009). However, prior to entering permanent cell cycle arrest, mutation-harboring cells transiently expand, giving rise to a spatially restricted lesion enriched for senescent cells. Once in full senescence, cells activate specialized SASP secretome (Coppe et al., 2008).

Several inflammatory cytokines and growth factors are part of SASP, and their signaling roles are being rapidly recognized in modulating biological processes, including normal embryonic development (Storer et al., 2013), cellular plasticity and reprogramming (Mosteiro et al., 2016; Ritschka et al., 2017), injury repair (Chiche et al., 2017; Demaria et al., 2014), and cancer progression (Capell et al., 2016; Herranz et al., 2015; Laherge et al., 2015; Ruhland et al., 2016; Yoshimato et al., 2013).

### Senescent Melanocytes Disrupt Hair Follicle Stem Cell Auiescence

It was considered how senescent melanocytes affect HF cell populations with active roles in hair regeneration: bulge SCs, hair germ (HG) progenitors and DP fibroblasts. Their transcriptomes were profiled by RNA-sequencing (RNA-seq) following cell sorting. Bulge and HG cells were isolated from Tyr-Nrms^{Q61K};K14-H2B-GFP mutant and K14-H2B-GFP control mice as GFP⁺/CD34⁺/Pcad^{lo} and GFP⁺/CD34^{neg}/Pcad^{hi} populations, respectively (Greco et al., 2009). Subset of DP fibroblasts was isolated from Tyr-Nras^{Q61K};Sox2-GFP mutant and Sox2-GFP control mice as GFP⁺; CD133⁺ population (Driskell et al., 2009). Bulge and DP cells were profiled at P30 and P56, when dorsal HFs in control, wildtype (WT) mice are in second anagen and telogen, respectively. HG progenitors were profiled at P56, since they exist only during telogen phase.

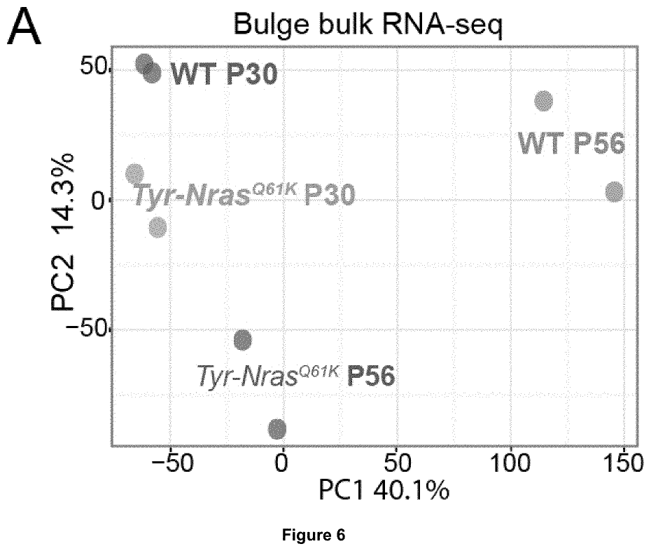

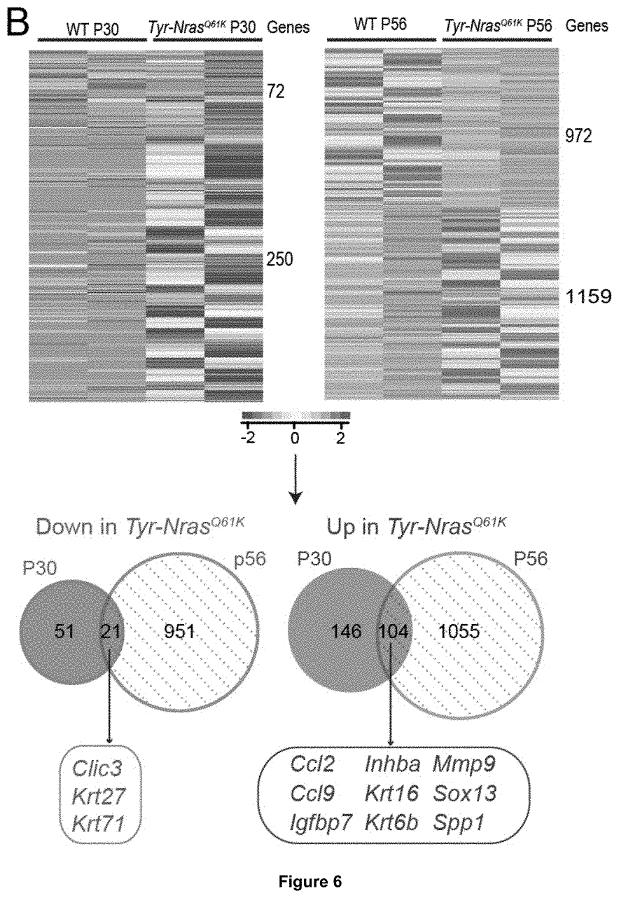

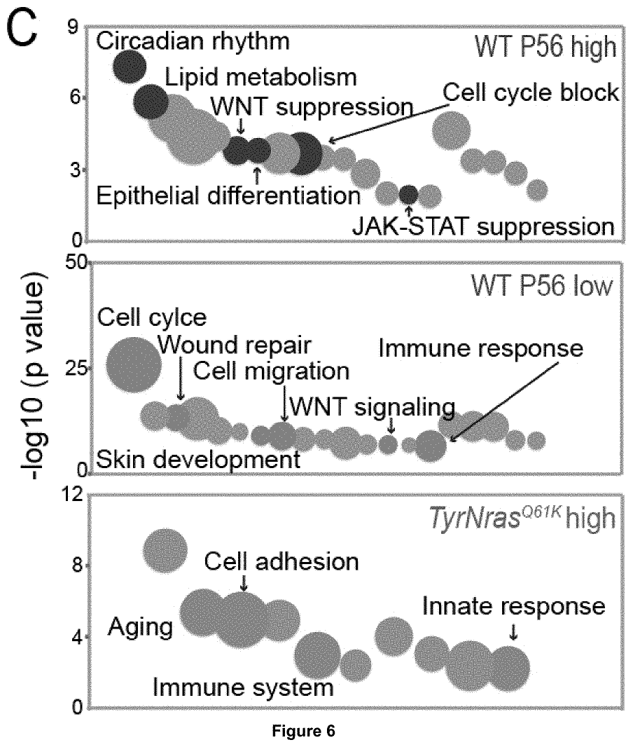

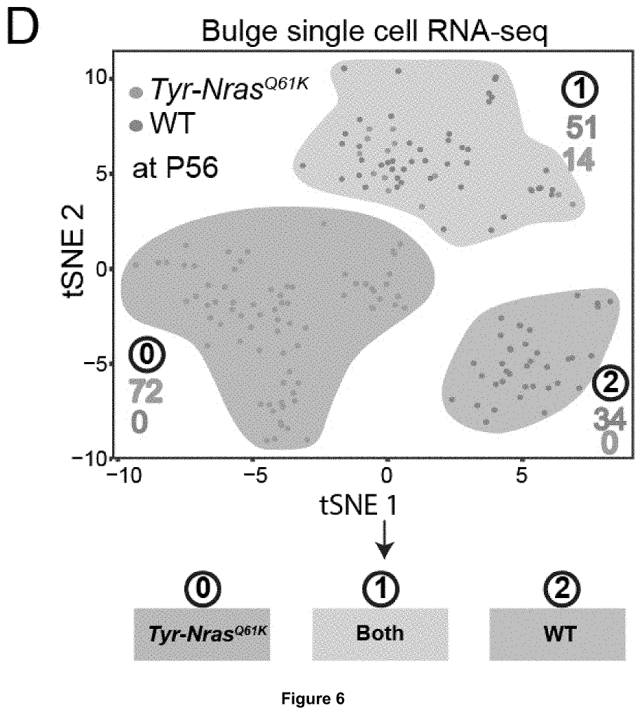

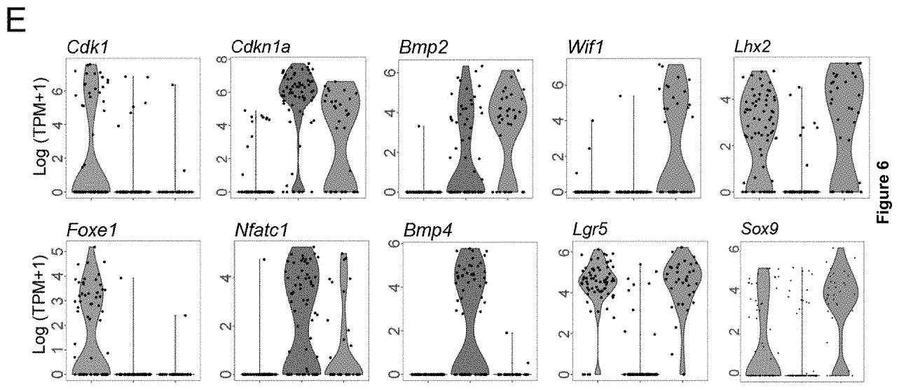

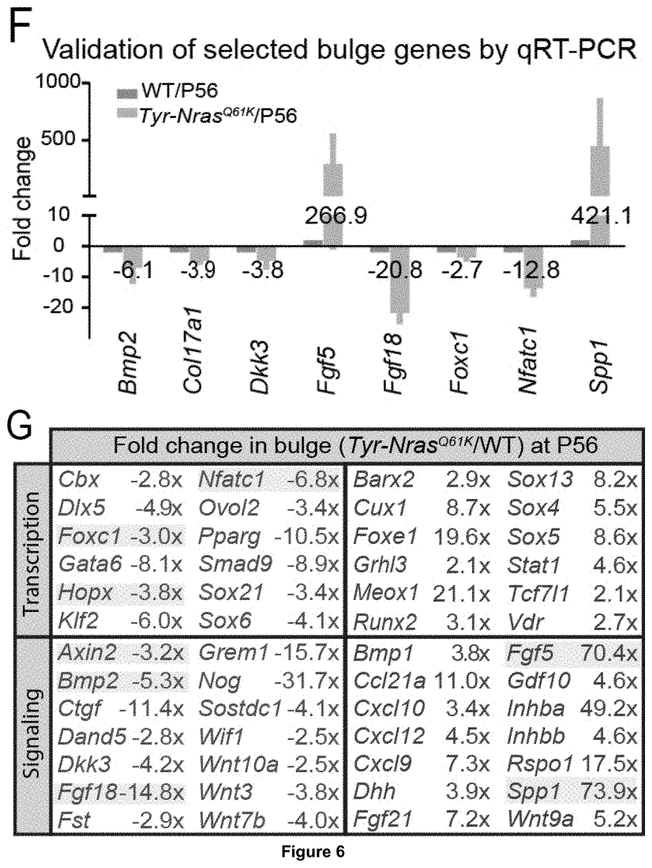

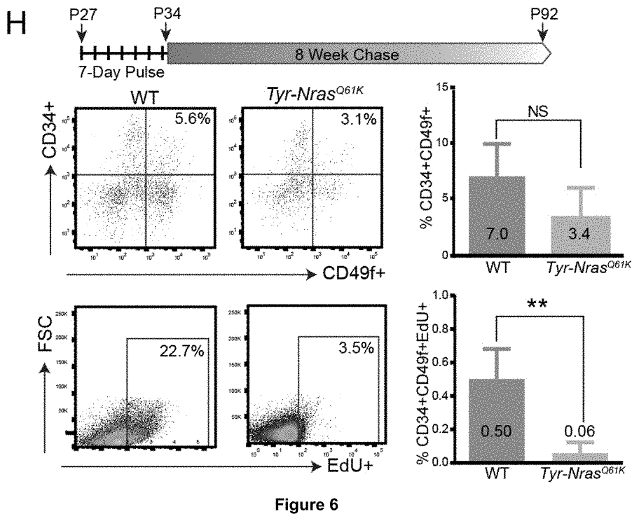

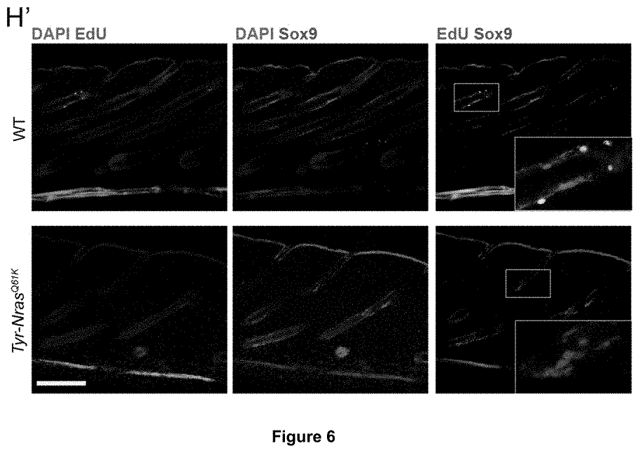

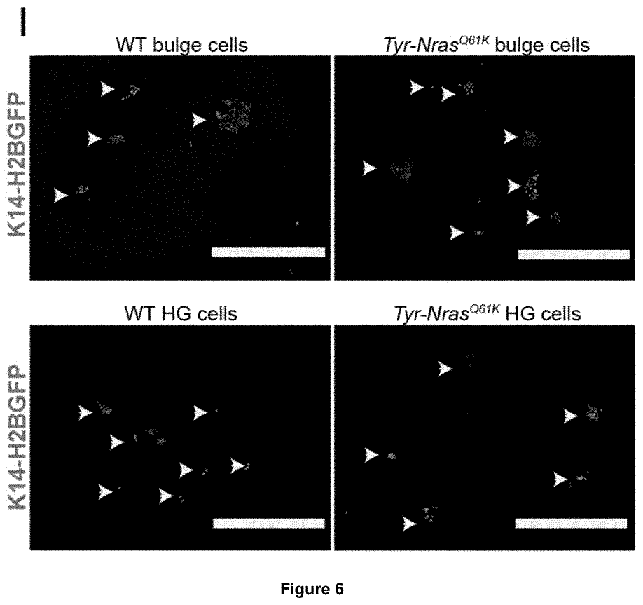

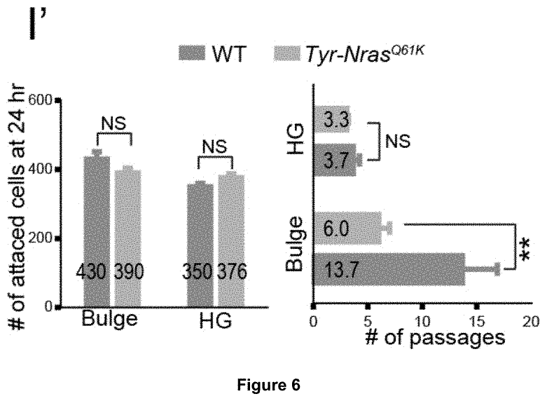

RNA-seq analysis on bulge SCs revealed prominent differences between Tyr-Nras^{Q61K} and WT mice at both time points (FIG. 6A). Multiple differentially expressed genes were identified (FIG. 6B). The largest differences were observed between P56 WT and mutant samples. P56 WT cells upregulate 973 genes, while P56 mutant cells upregulate 1,159 genes. Gene ontology (GO) analysis showed enrichment of WT cells for gene categories, including cell cycle block, circadian rhythm, WNT and JAK-STAT suppression (FIG. 6C). Conversely, analysis on mutant cells showed enrichment for cell cycle, cell migration, WNT signaling and skin development gene categories (FIG. 6C). These GO signatures indicate that WT bulge SC's are quiescent at P56, consistent with HFs being in telogen phase. It also suggests that Tyr-Nras^{Q61K} bulge SCs loose quiescence. However, alternative to this is that RNA-seq signature of Tyr-Nras^{Q61K} samples is a composite of telogen and anagen bulge SCs, since P56 mutant skin contains numerous ectopic anagen HFs.

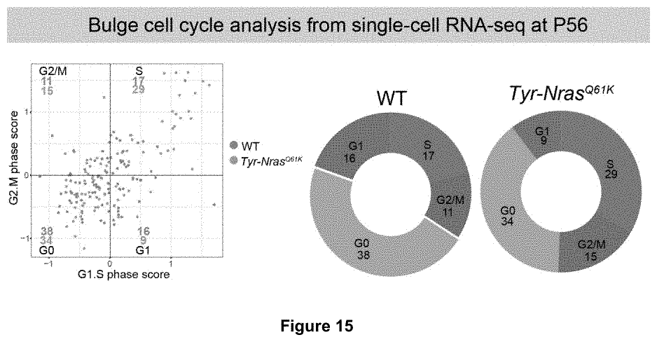

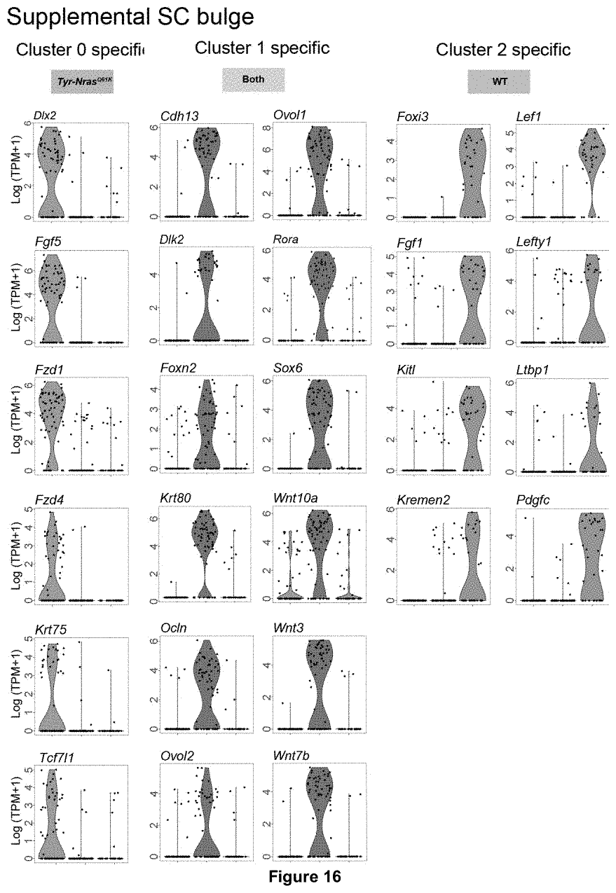

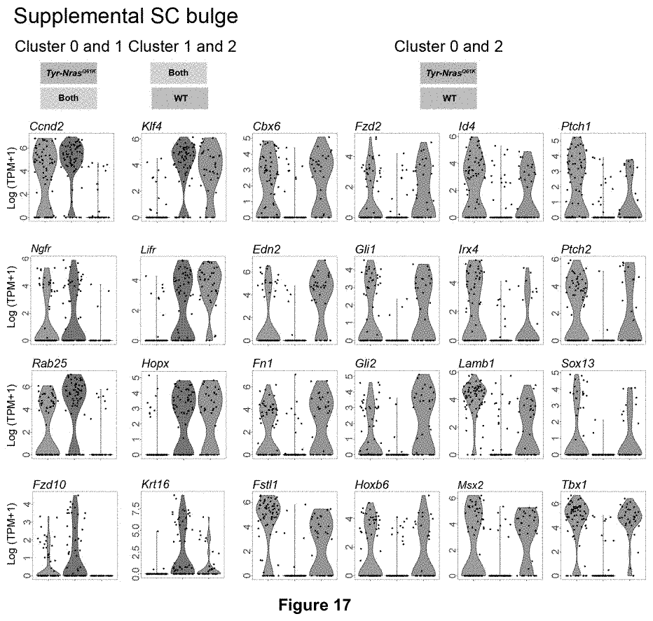

To address these possibilities, bulge SCs were compared between P56 Tyr-NrasQ^{61K} and WT mice on single-cell RNA-seq. Analysis shows that WT bulge SCs consist of two distinct types (top-right and bottom-right clusters in FIG. 6D). Gene profiling indicates that both SC types are quiescent, expressing high levels of cell cycle inhibitor Cdkn1a, quiescence markers Nfatc1 (Horsley et al., 2008), Hopx (Takeda et al., 2013) and signaling inhibitor Bmp2 (FIG. 6E). Intriguingly, mutant bulge SCs dramatically alter their composition. Many SCs become entirely new type (bottom-left cluster in FIG. 6D), while quiescent SCs types either completely disappear (bottom-right cluster) or become largely depleted (top-right cluster). Loss of quiescence is evident on cell cycle analysis (FIG. 15) and gene expression, Mutant-specific SCs downregulate Cdkn1a, Nfatc1, Hopx, Bmp2/4 and upregulate cell cycle activator Cdk1 and Hedgehog pathway target Foxe1 (Eichberger et al., 2004). At the same time, they share many markers with WT SCs from the bottom-right cluster: transcriptional factors Lhx2 (Folgueras et al., 2013), Sox9 (Vidal et al., 2005), Thx1 (Chen et al., 2012), G-protein coupled receptor Lgr5 (Jaks et al., 2008) and Hedgehog pathway components Glil1/2, Ptch1/2, but are largely distinct from SCs in the top-right cluster of FIG. 6D (see FIGS. 6E, 16 and 17). The later express several unique transcriptional factors Foxn2, Ovol1/2, Sox6 and canonical WNT ligands Wnt3/7b/10a. Given that P56 Tyr-Nras^{61K} skin contains many telogen HFs, disappearance of bottom-right cluster SCs is consistent with the dramatic loss of quiescence by telogen bulge SCs in mutant mice. Marker similarities between bottom-right cluster SCs in WT and bottom-left cluster SCs in Tyr-Nras^{Q61K} mice suggest that former quiescent SCs transition into activated state in the presence of senescent melanocytes.

Next, loss of quiescence was confirmed in functional assays. A pulse-chase experiment was performed on bulge SCs. Mice were pulsed with EdU between days P27-P34, when WT HFs are in early anagen and their SCs are proliferative (Hsu et al., 2011), and then chased till day P92. On cytometric analysis, the total number of bulge SCs in Tyr-Nras^{Q61K} mice did not significantly differ from WT, however, there was prominent loss of EdU-retaining SCs (n=4) (FIG. 6H). Depletion of label-retaining SCs in mutants was confirmed on co-staining with Sox9 (FIG. 6H′). A long-term clonogenic assay was performed on sorted bulge SCs. While the attachment ability of Tyr-Nras^{Q61K} bulge SCs was similar to that of WT SCs, their serial passaging potential was severely compromised: mutant SCs supported on average 6 passages (n=3) compared to 13.7 passages for WT SCs (n=3) (FIGS. 6I and 6I′). At the same time, attachment rate and passaging potential did not differ between mutant (n=3) and WT short-term HG progenitors (n=3).

It was then asked what signaling changes accompany loss of quiescence by bulge SCs in Tyr-Nras^{Q61K} mice. To address this, bulk RNA-seq data was analyzed, which has higher depth of coverage compared to single-cell data. First, it was confirmed that quiescence markers from single-cell analysis, Nfatc1 and Hopx, were also downregulated in P56 Nras^{Q61K} vs. WT SCs on bulk analysis. Other quiescence markers were also downregulated in Nras^{Q61K} SCs: Axin2 (Lim et al., 2016), Col17a1 (Matsumura et al., 2016), Fgf18 (Kimura-Ueki et al., 2012) and Foxc1 (Lay et al,, 2016; Wang et al., 2016) (FIGS. 6F and 6G). In addition, Nras^{Q61K} SCs dokvnregulate multiple signaling regulators for BMP pathway: Bmp2, Fst, Grem1, Dand5, Nbl1, Nog, Smad9, Sostdc1, WNT pathway: Dkk3, Dkkl1, Fzd2/3/7/9, Wnt10a/3/7b, Wif1, as well as Ctgf, Fgf1, and Hhip. Conversely, they upregulate TGFβ pathway members: Bmp1, Gdf10, Inhba, Inhbb, Tgfb1, WNT pathway factors: Fzd4/5, Rspo1, Wnt9a, as well as Fgf5, Fgf21, Ccl21a, Cxcl9/10/12, Il1f5 and Spp1 (FIG. 6G). Among the above factors, Spp1 (aka osteopontin) showed the largest fold change (73.9×) and high expression values in P56 Nras^{Q61K} compared to WT SCs. In fact, it was also highly expressed by P30 Nras^{Q61K} SCs and virtually not expressed by P30 SCs. This suggests that Spp1 is specifically upregulated in HF SCs of Tyr-Nras^{Q61K} mice.

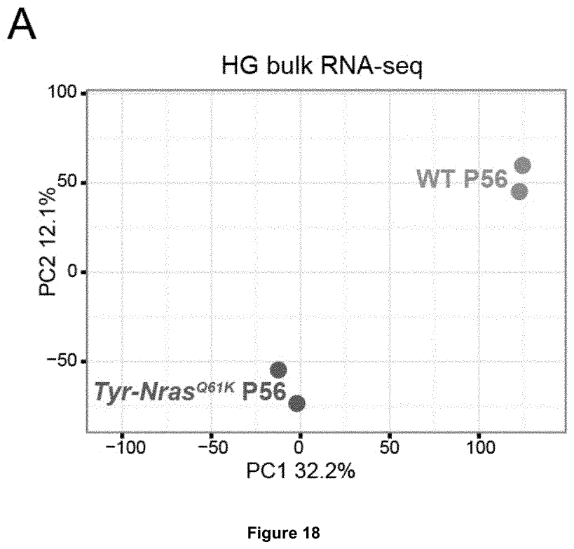

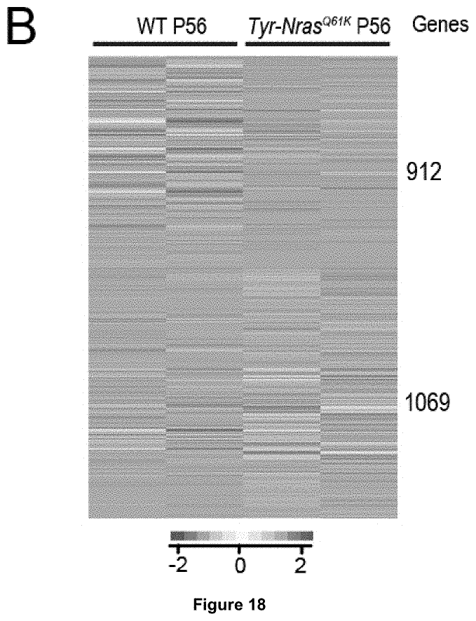

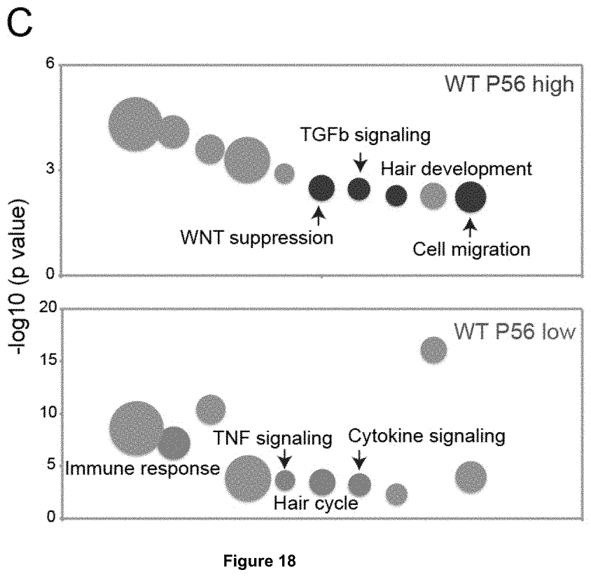

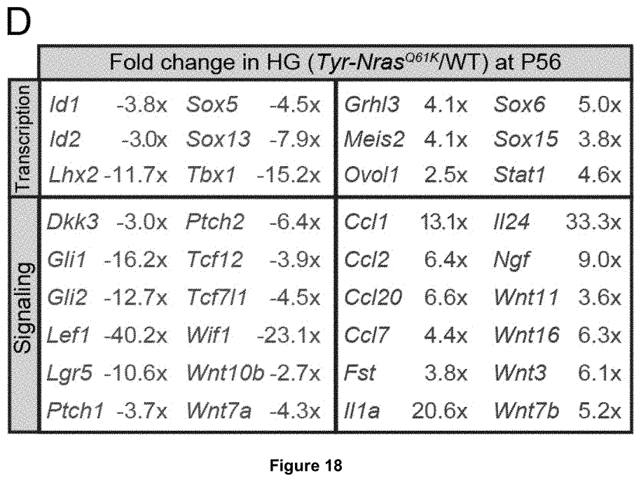

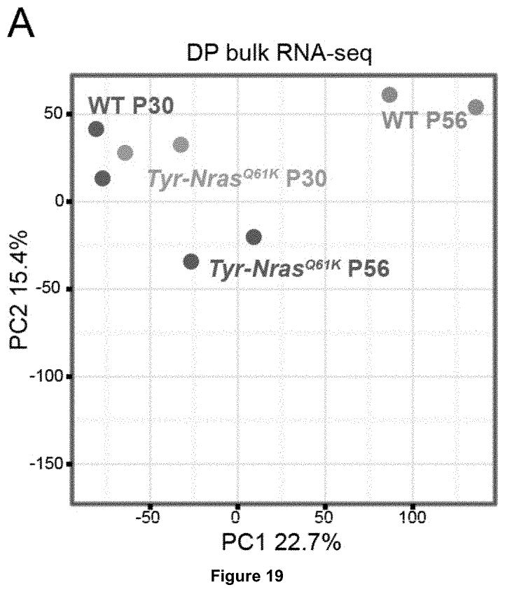

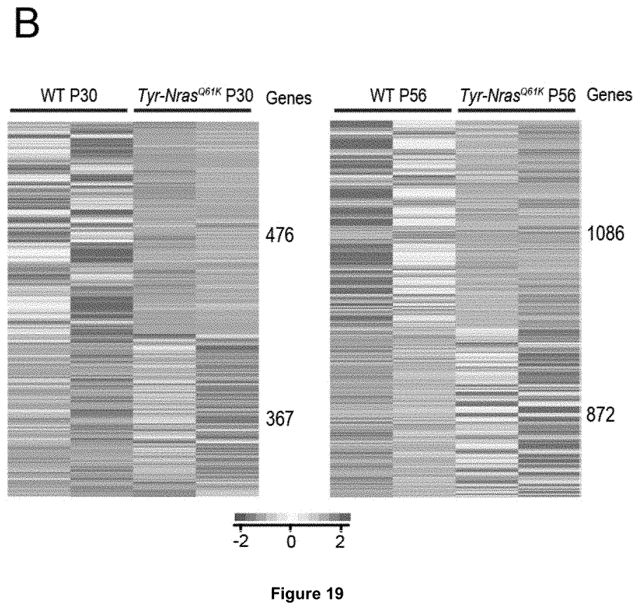

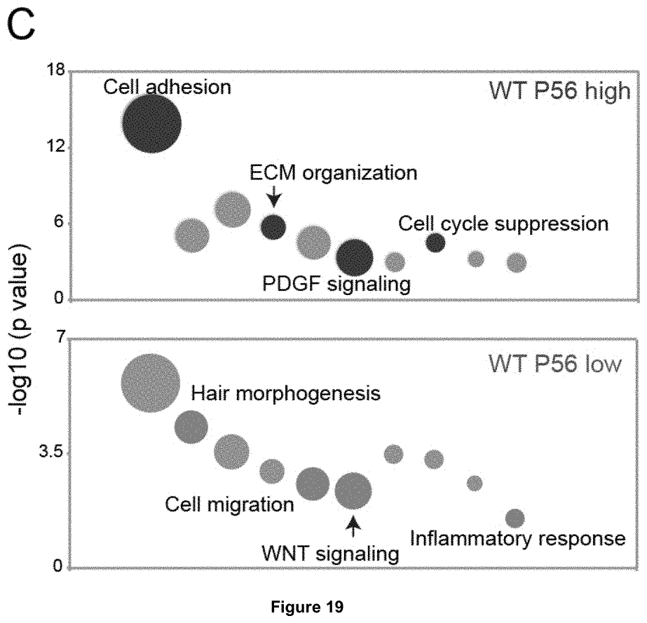

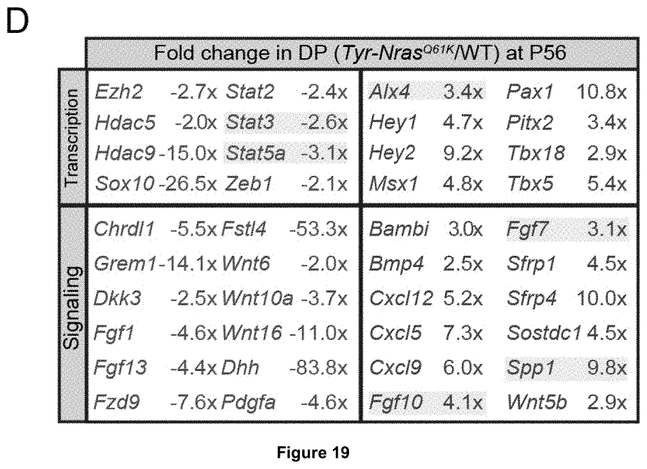

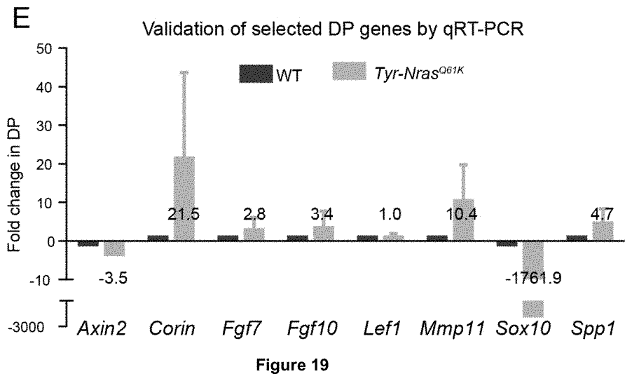

Compared to WT control, P56 Nras^{Q61K} HG progenitors upregulate several transcriptional factors, including Grh13, Meis2, Ovol1, Sox6/15, canonical (Wnt3/3a/7b/10a) and non-canonical WNT ligands (Wnt5a/5b/11/16), multiple cytokines Ccl1/2/7/20/27a/27b, interleukins I11a/1 b/1f5/1f9/6/24/34 and chemokines Cxcl9/16, as well as Fst and Ngf. They downregulate transcriptional factors Id1/2/4, Lhx2, Sox5/13, Tbx1, multiple WNT pathway components: Dkk3, Fzd2/3/7/8, Lgr4/5, Lef1, Lrp6, Tcf7l1/12, Wnt7a/10h, Wif1, and Hedgehog pathway members Glil1/2, Ptch1/2 (FIGS. 18A-18D). This RNA-seq signature is consistent with complex changes in WNT pathway, inhibitory changes in Hedgehog pathway, and prominent activation of inflammatory signaling. P56 Nras^{Q61K} DP fibroblasts upregulate transcriptional factors Alx4, Hey1/2, Msx1/2, Pax1, Pitx2, Sox18, Tbx5/18, BMP pathway components: Bmp4, Bambi, Id1, Sosidc1, WNT pathway components: Fzd4/10, Rspo1/3/4, Sfrp1/4, Wnt5b, as well as Fgf7/10, Spp1 and cytokines Cxcl1/5/9/12/14. They downregulate transcriptional and epigenetic factors Ezh2, Hdac5/9, Stat2/3/5a, Zeb1, BMP components: Chrdl1, Grem1, WNT components Dkk3, Ezd9, Wnt6/10a/16 as well as Dhh and Pdgfa (FIGS. 19A-19E). This RNA-seq signature highlights complex changes in WNT and BMP ligand and antagonist production, activation of hair growth-promoting FGF ligands, as well as cytokines, including osteopontin production.

### Immune Cells Augment Secretome of Nevus Melanocytes

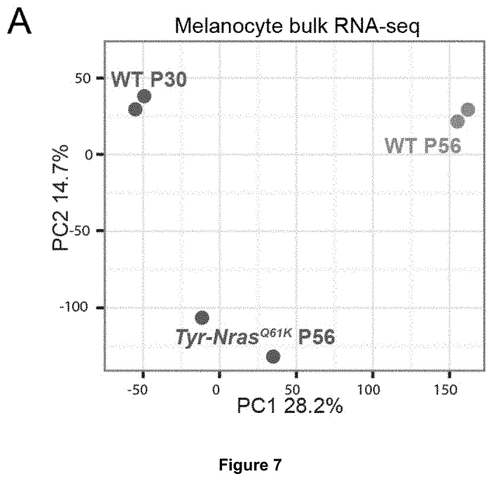

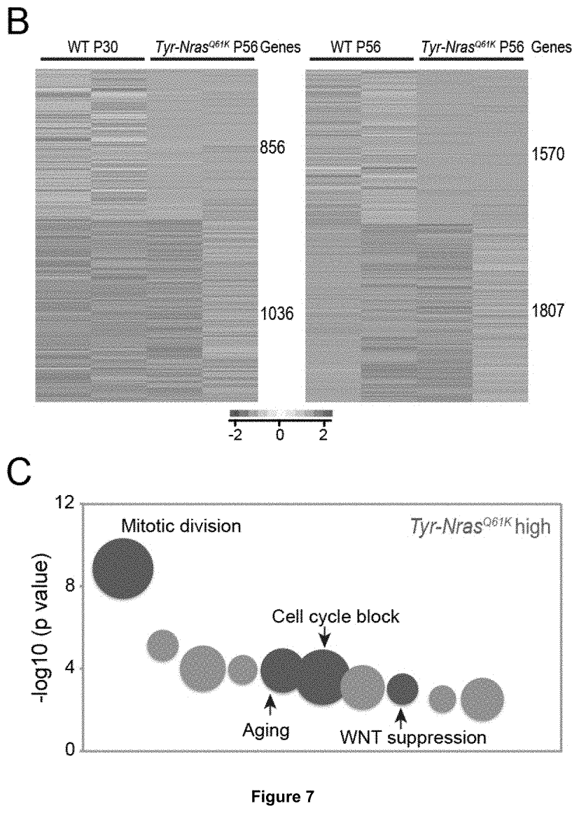

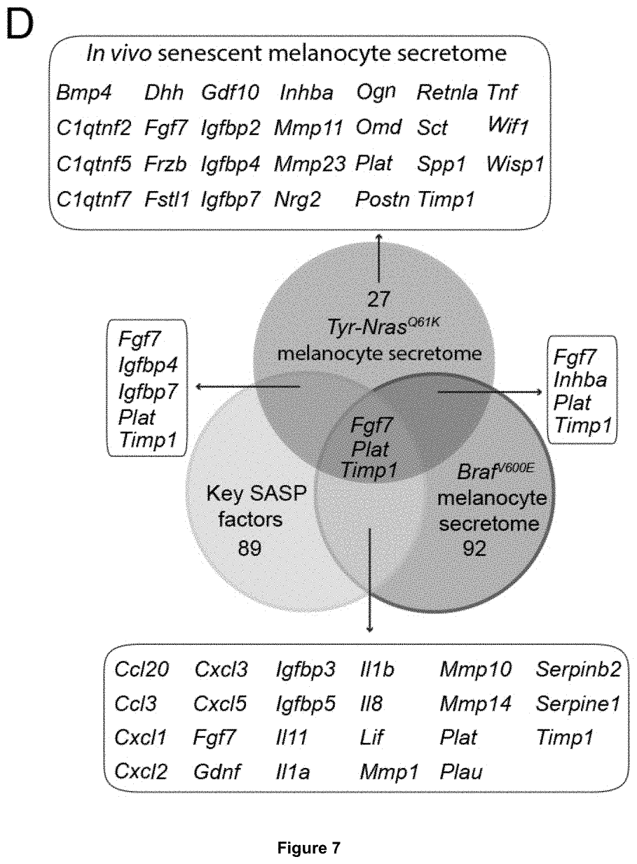

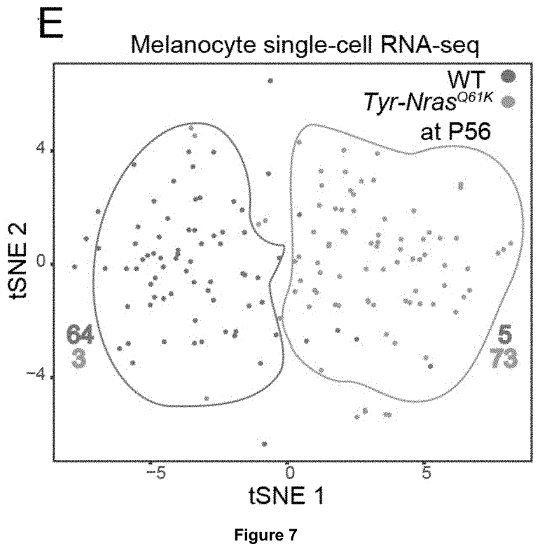

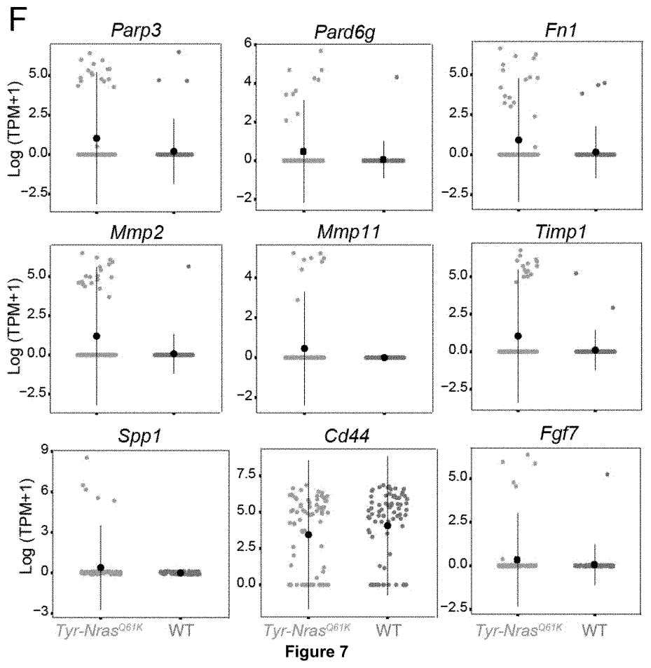

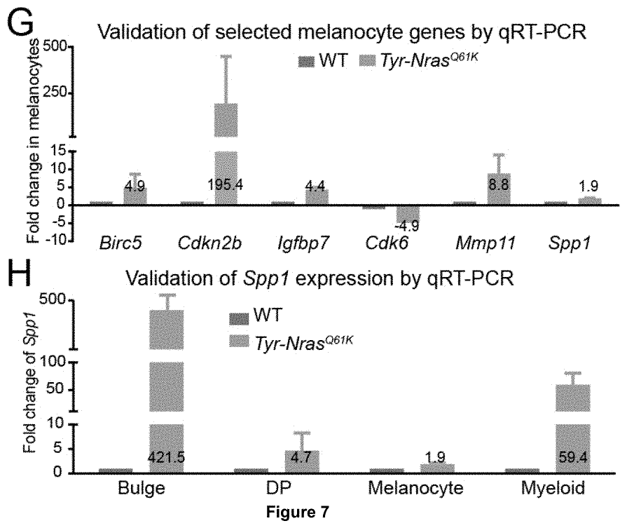

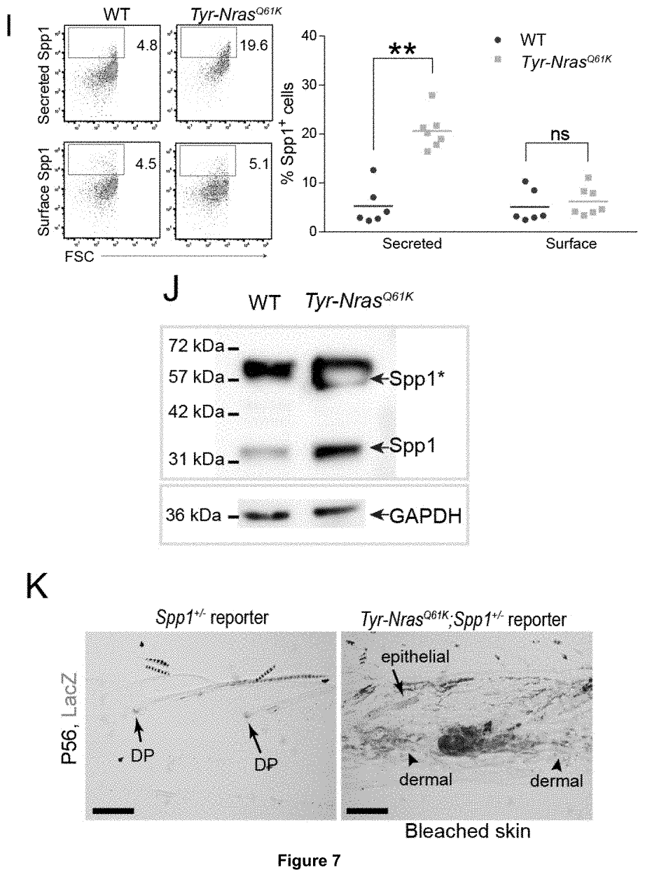

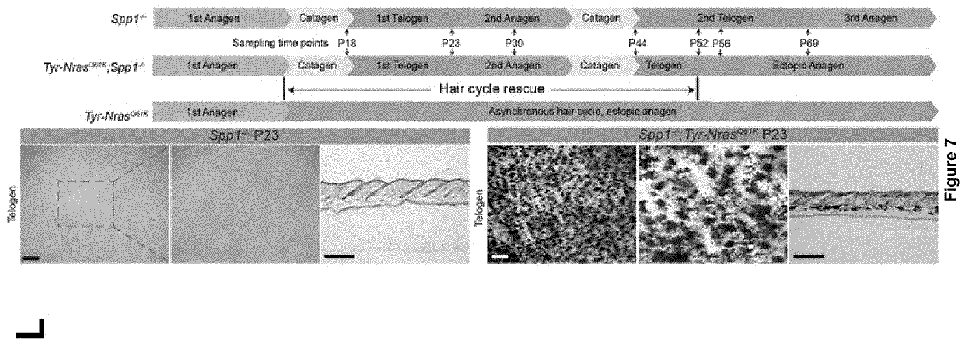

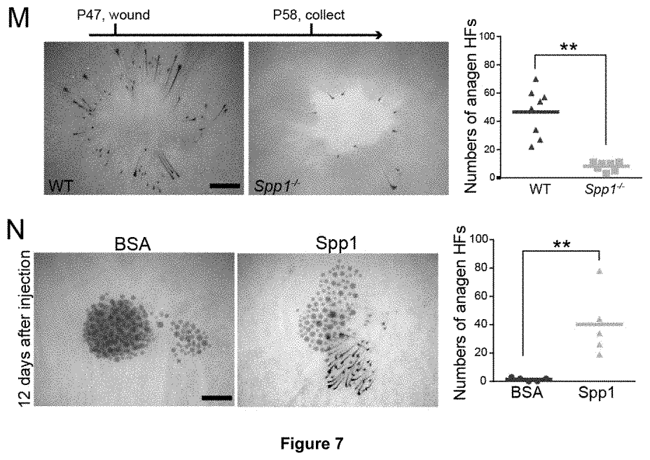

A melanocyte lineage was isolated as tdTomato⁺ cells from Tyr-Nras^{Q61K};Tyr-CreER^{T2};tdTomato mutant and Tyr-CreER^{T2};tdTomato control mouse skin. Transcriptome of P56 mutant samples was then compared to both P30 anagen and P56 telogen WT samples (FIGS. 7A and 7B). This strategy identified 598 mutant-specific upregulated genes, and excluded genes regulated in melanocyte lineage as the function of normal hair cycle. Mutant-specific genes were enriched for GO terms, including aging, WNT suppression, cell cycle block and mitotic division (FIG. 7C). Consistent with mutant melanocytes undergoing OIS, they strongly upregulate tumor suppressors Cdkn1b (aka p15), Lzts1, as well as Cdkn3, H2afx and a number of mitosis-associated genes, including Aurka/b, Cdca3/8, Cdc20/25c, Cenpa, Mad2l1, Ncaph, Knstrn, Plk1, Psrc1, Reep4. Paradoxical upregulation of the latter gene set is consistent with extended mitotic arrest, mitotic slippage and multinucleation by OIS melanocytes (Dikovskaya et al., 2015). Twenty-seven secretory signaling factors uniquely upregulated in nevus melanocytes were identified (FIG. 7D). These include BMP pathway members: Bmp4, Fstl1, WNT pathway members: Frzb, Wif1, Wisp1, IGF regulators Igfbp2/4/7, as well as Dhh, Fgf7, Spp1 and Tnf. Intriguingly, there was little overlap between the secretome of Tyr-Nras^{Q61K} mouse nevus melanocytes in vivo and the secretome of BRAF^{V600E} induced human senescent melanocytes in vitro (Pawlikowski et al., 2013), or the core SASP factors (Andriani et al., 2016; Coppe et al., 2010; Coppe et al., 2008; Freund et al., 2010). Tyr Nras^{61K} nevus melanocytes distinctly did not upregulate multiple CC and CXC chemokines or interleukins, otherwise characteristic of in vitro SASP (FIG. 7D) Upregulation of select cell cycle inhibitors and secretome genes in P56 Tyr-Nras^{Q61K} nevus melanocytes was also confirmed on single-cell RNA-seq (FIGS. 7E and 7F). Together, this data shows that senescent melanocytes in Tyr Nras^{Q61K} skin upregulate a unique secretome that is largely distinct from an in vitro senescent cell secretome.

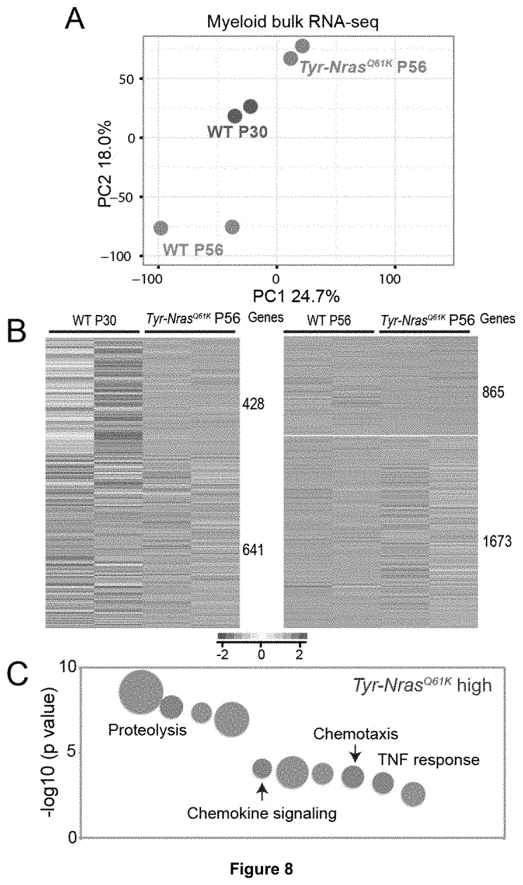

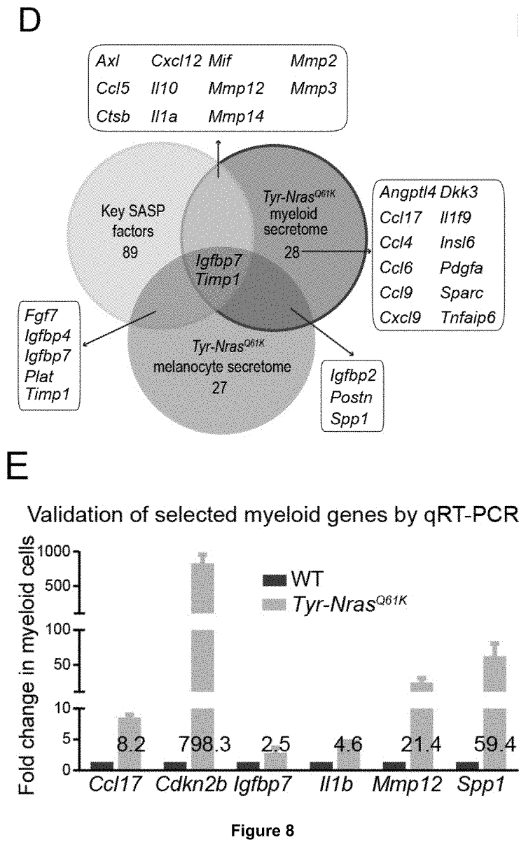

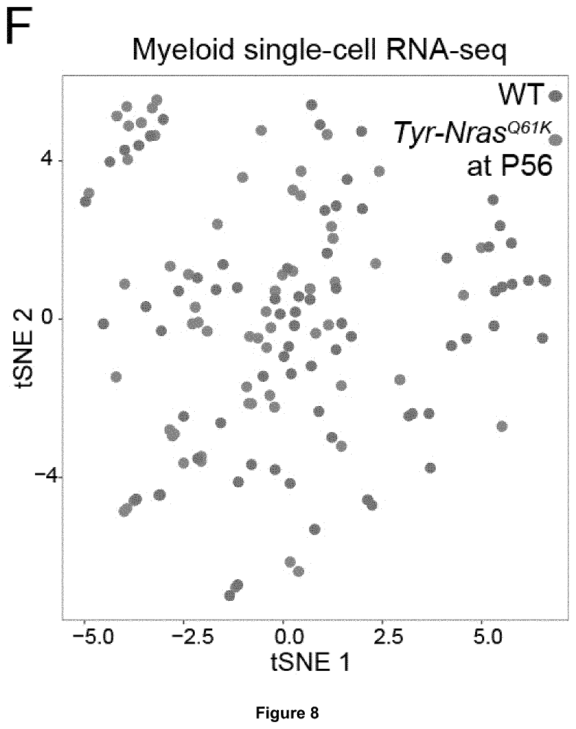

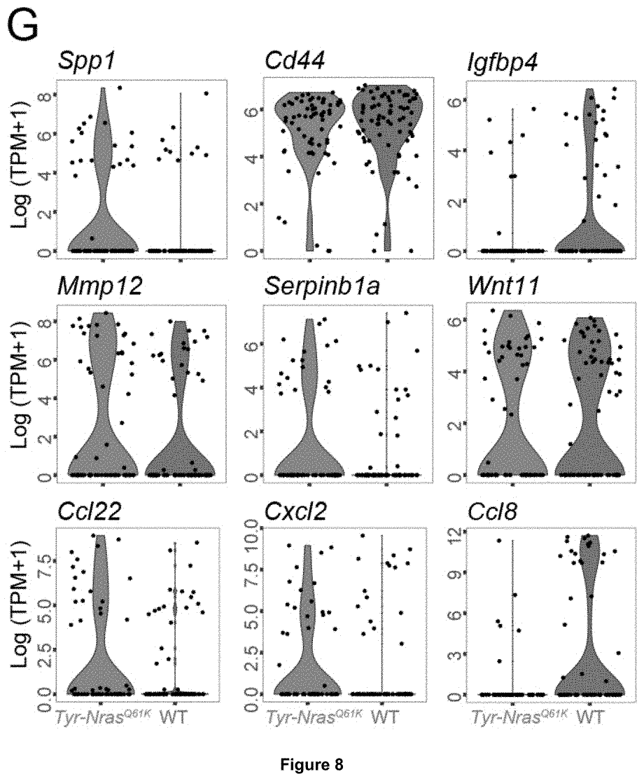

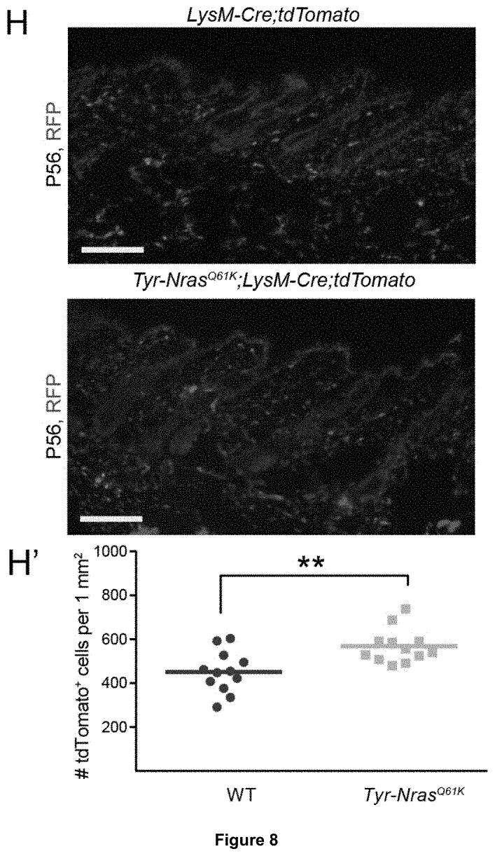

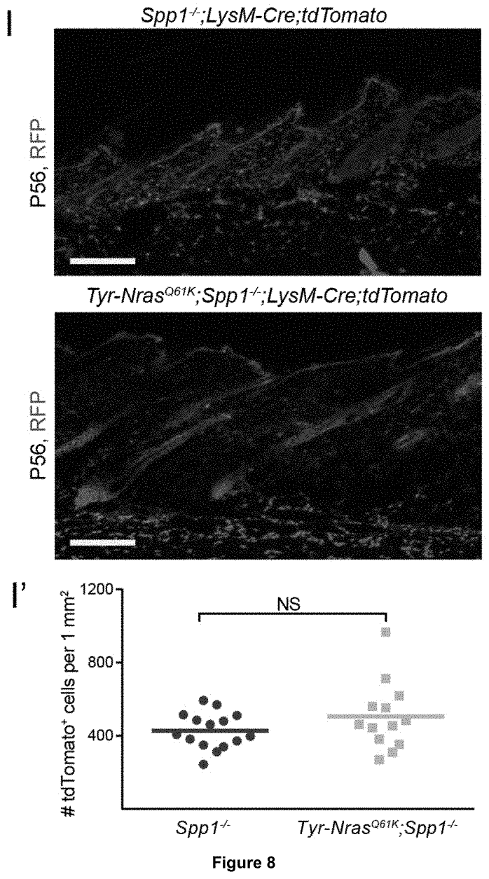

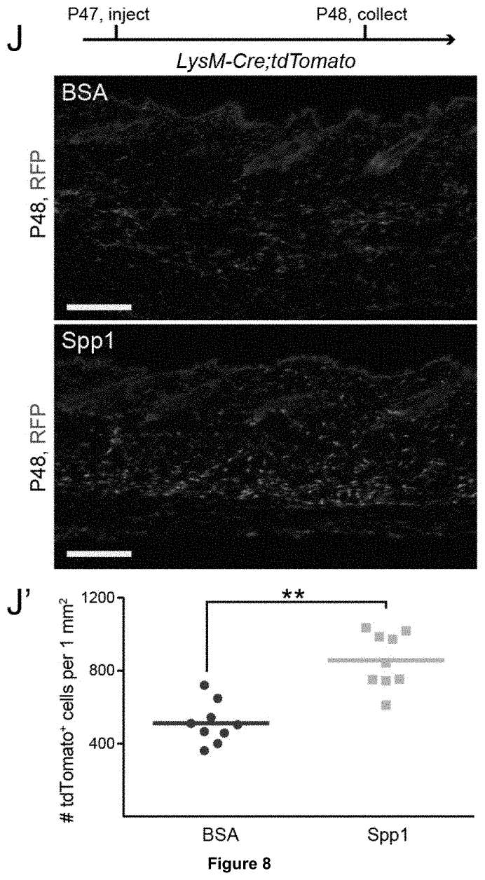

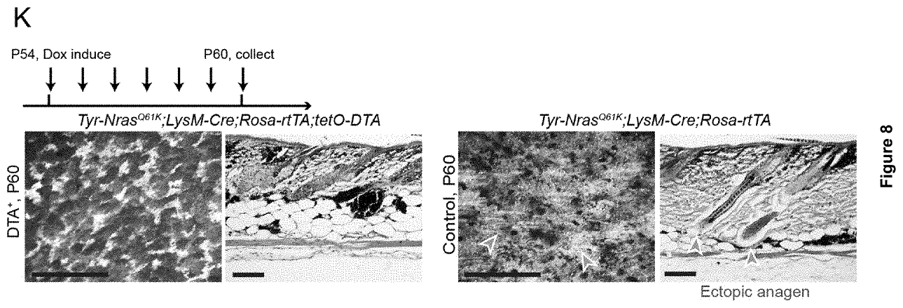

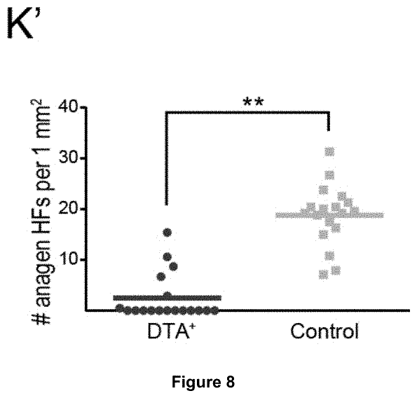

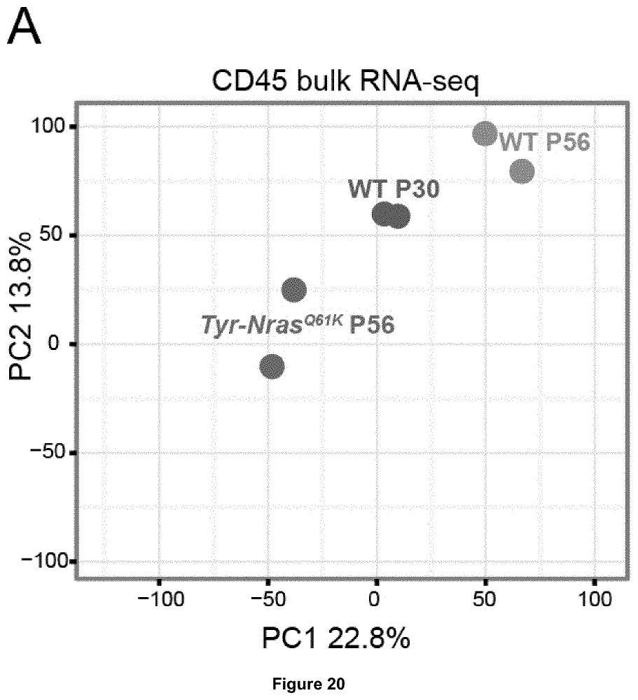

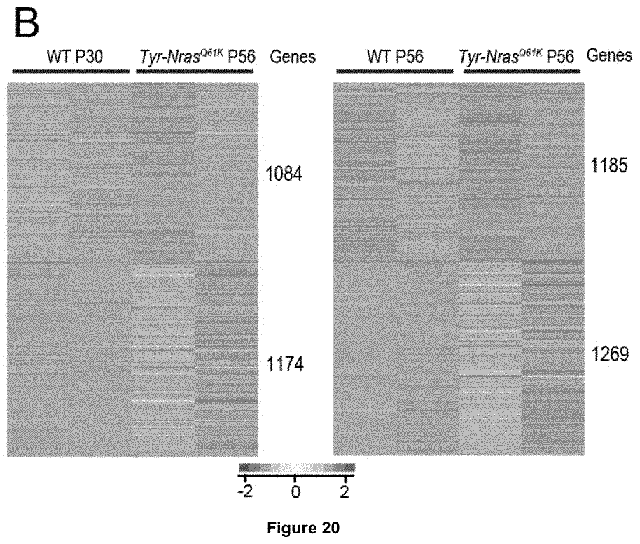

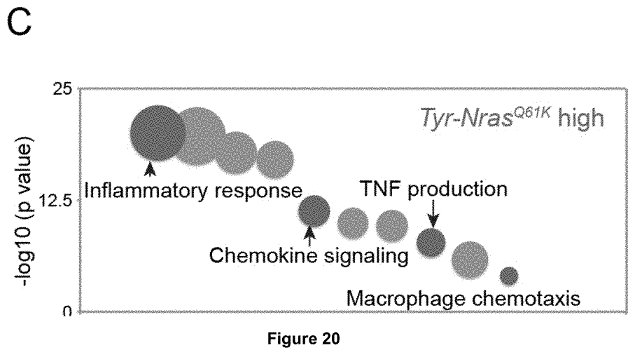

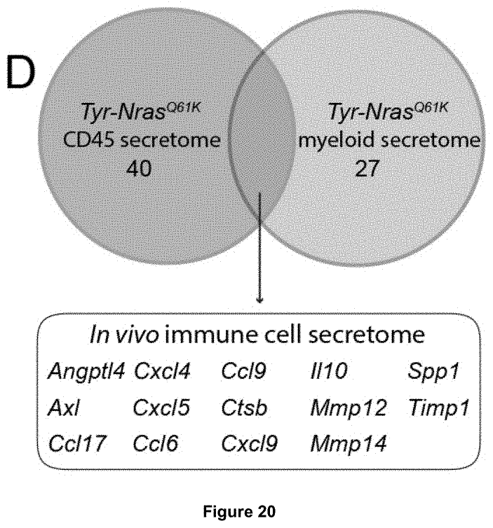

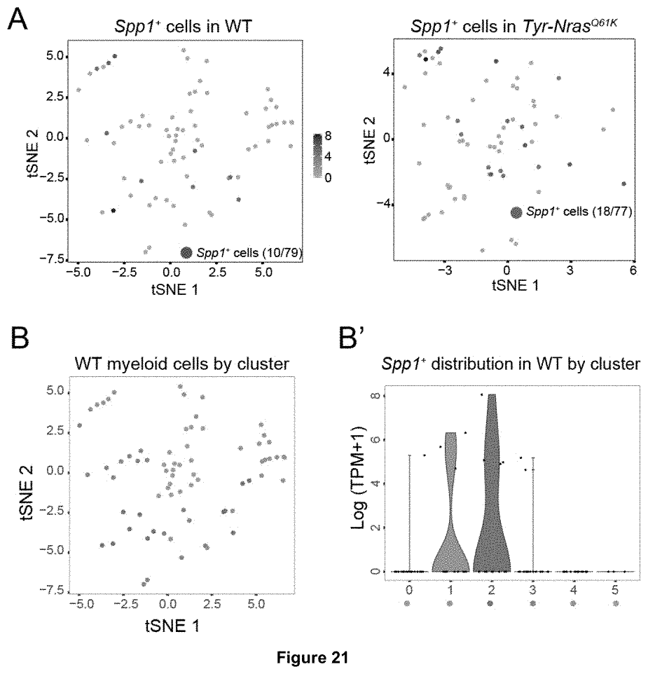

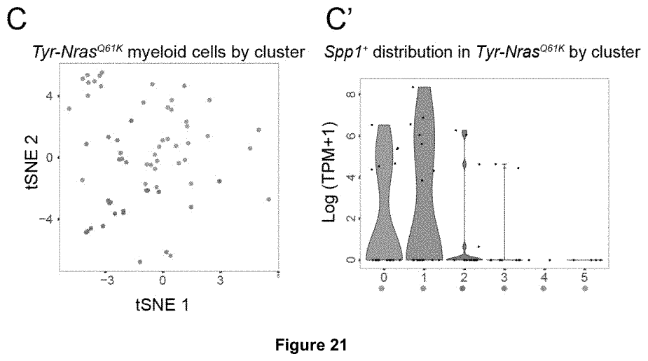

Considering a rapidly emerging role for immune cell signaling in hair cycle activation (Ali et al., 2017; Amberg et al., 2016, Castellana et al., 2014; Chen et al., 2015; Gay et al., 2013; Lee et al., 2017; Wang et al., 2017b), experiments were conducted to sec if and how immune cell secretome is altered in the nevus skin. Transcriptomes were profiled of all skin-resident CD45-expresing hematopoietic cells (FIGS. 20A-20D) and tdTomato⁺ myeloid cells isolated based on LysM-Cre;tdTomato labeling (FIGS. 8A and 8B). To identify mutant-specific genes, the same RNA-seq analysis strategy was used as with melanocytes and compared P56 mutant cells to both P30 and P56 WT cells. Transcriptome of mutant-specific myeloid cells was enriched for GO terms, including chemotaxis, proteolysis and chemokine signaling (FIG. 8C). Myeloid cells in nevus skin prominently upregulate secreted factors, that belong to SASP—a feature validated by qRT-PCR (FIG. 8E) and single-cell RNA-seq (FIGS. 8F and 8G). Specifically, they upregulate CC chemokines Ccl4/5/6/9/17, CXC chemokines Cxcl19/12, interleukins Il1a/1f9/10 and matrix metalloproteinases Mmp2/3/12/14, otherwise lacking in the secretome of nevus melanocytes (FIG. 8D). In addition, similar to nevus melanocytes, they upregulate IGF regulators Igfbp2/7 as well as Postn, Spp1 and Timp1 (FIGS. 8D and 21).

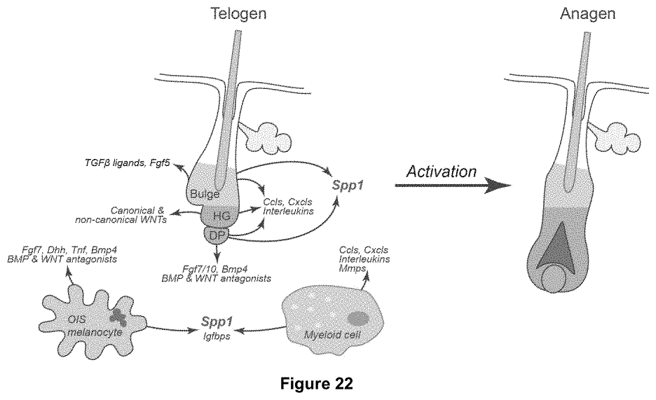

Taken together, the combined secretome of senescent melanocytes and myeloid cells distinctly enriches signaling environment of nevus skin for multiple inflammatory pathway ligands (FIG. 22). Senescent melanocytes and myeloid cells jointly express Spp1, while myeloid cells express multiple CC and CXC chemokines and interleukins. In addition, senescent melanocytes express several BMP and WNT pathway modulators, as well as Dhh, Fgf7 and Tnf. In turn, these extra-follicular signaling activities induce changes in telogen HF compartments, including DP, HG and bulge SCs. All three compartments jointly upregulate several CC and CXC cytokines and interleukins. DP and bulge cells also prominently upregulate Spp1. Additionally, DP cells upregulate Fg17/10, Bmp4 and select secreted BMP and WNT antagonists, while HG cells upregulate several canonical and non-canonical WNT ligands. As the result of these senescent melanocyte-initiated signaling changes, bulge SCs loose quiescence and hair cycle entry by telogen HFs becomes prominently hyper-activated.

### Osteopontin Signaling Mediates Hair Cycle Activation in Mouse Skin

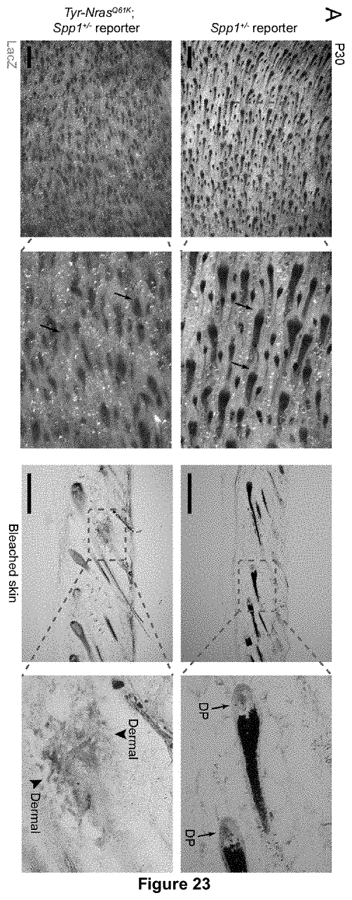

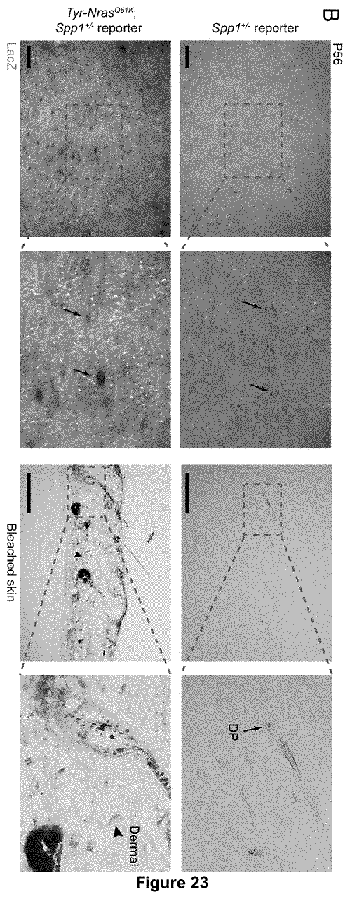

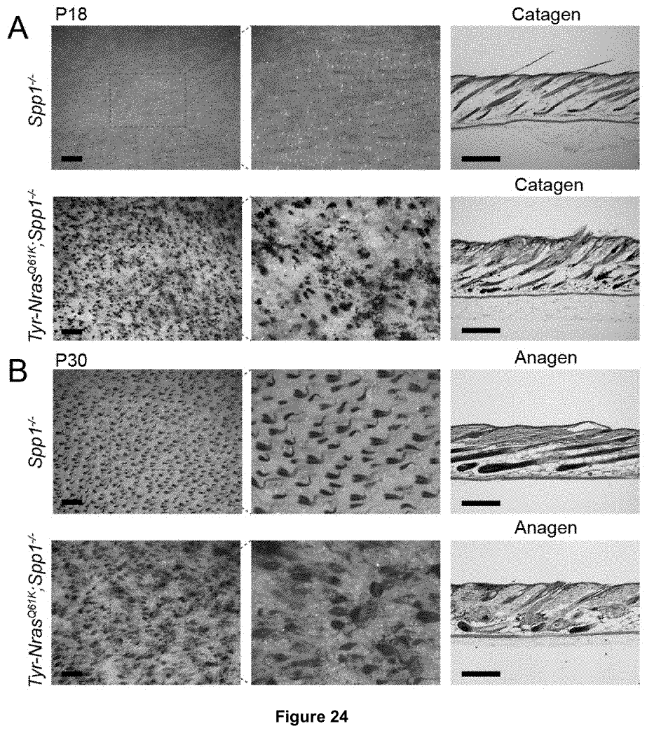

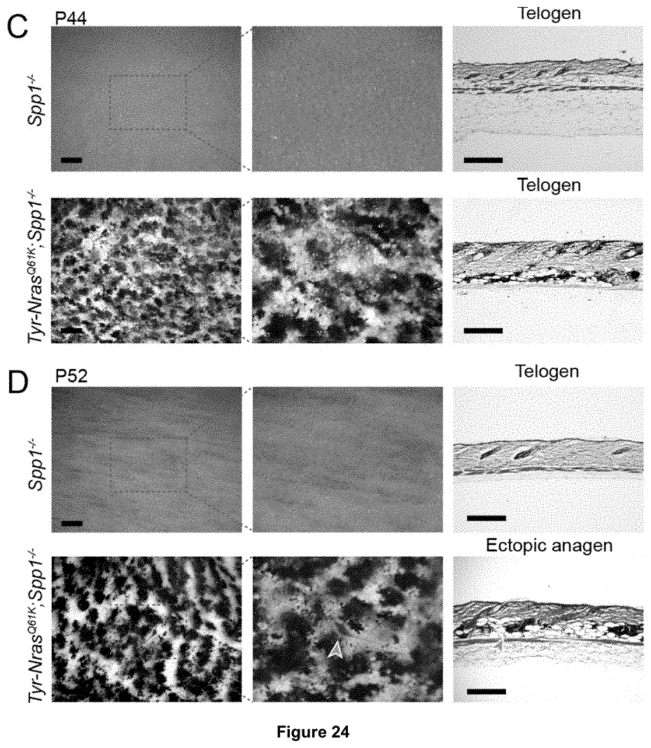

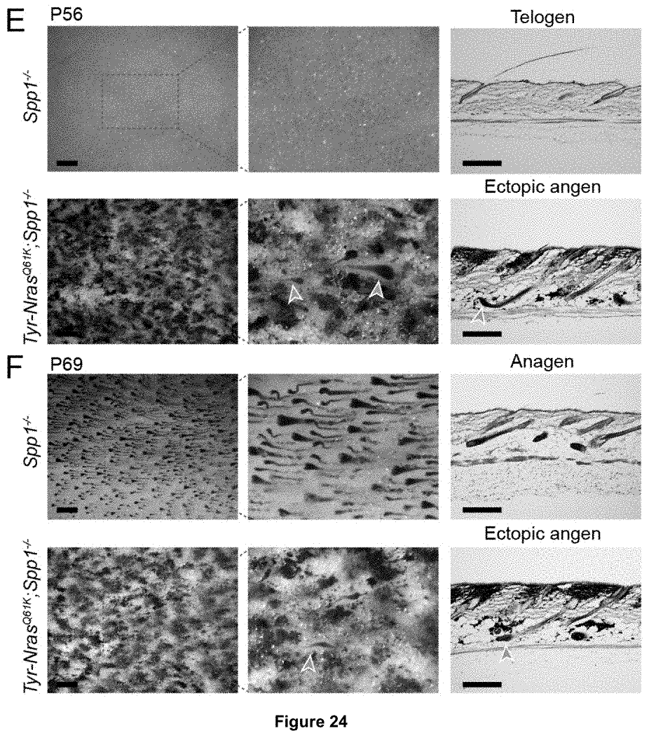

A transcript for osteopontin was one of the most prominently upregulated signaling factors in multiple nevus skin cell types (FIGS. 7H and 22). It was confirmed that osteopontin is increased in Tyr-Nras^{Q61K} skin at the protein level. Cytometric analysis on total skin showed significant increase in osteopontin-secreting cells in P56 Tyr-Nras^{Q61K} (n=4) vs. WT mice (n=4) (FIG. 7I). Consistently, higher osteopontin levels (both nascent ˜35 kDa and modified ˜65 kDa forms) were detected in P56 mutant skin by western blot (FIG. 7J) and on immunostaining, with multiple cell types, including HF epithelial cells, immune cells and dermal melanocytes being positive. Broad increase in the distribution of osteopontin-expressing skin cells was also confirmed on lacZ staining in Tyr-Nras^{Q61K};Spp1^{+/-} vs. control Spp1^{+/-} reporter mice (FIGS. 7K and 23).

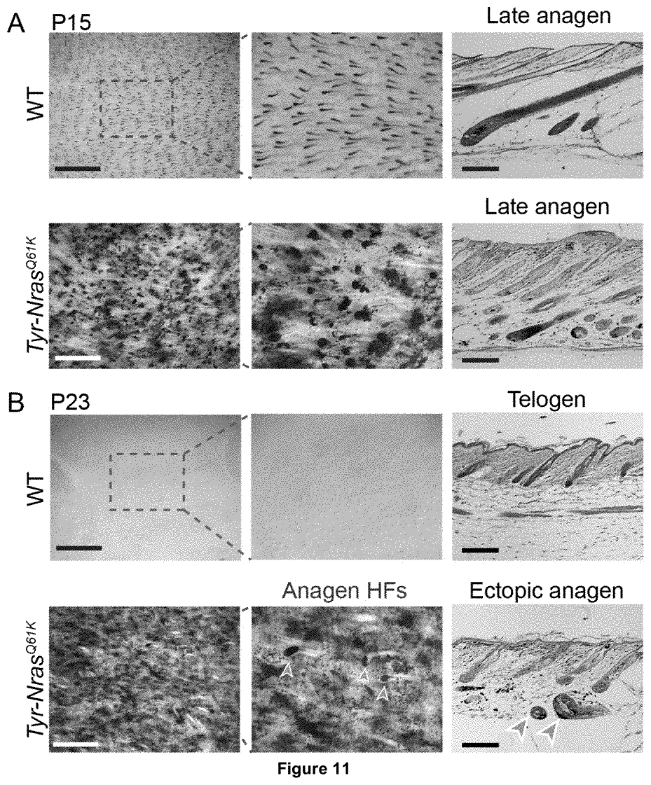

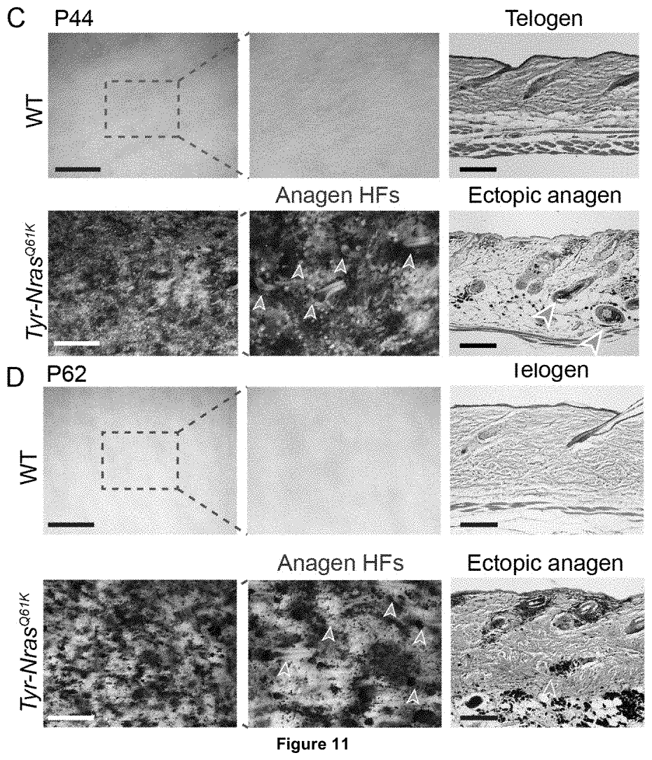

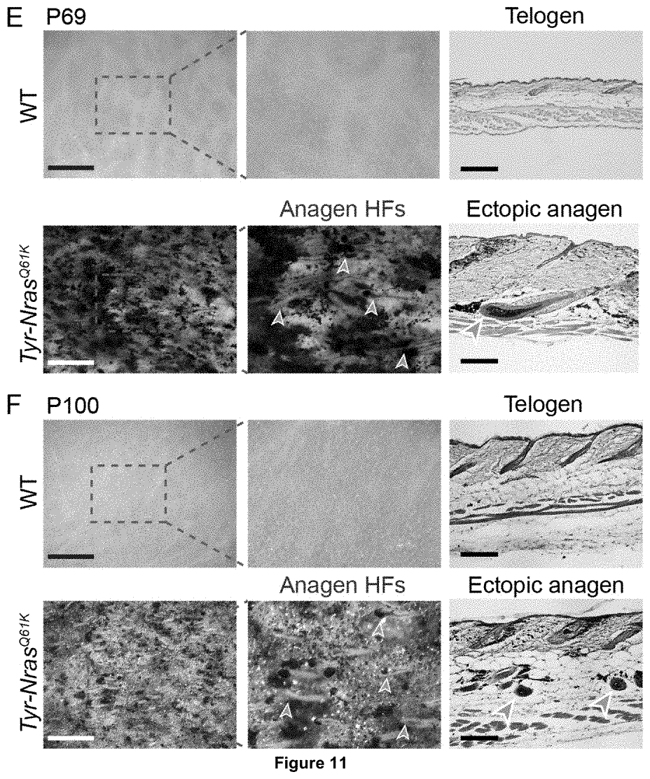

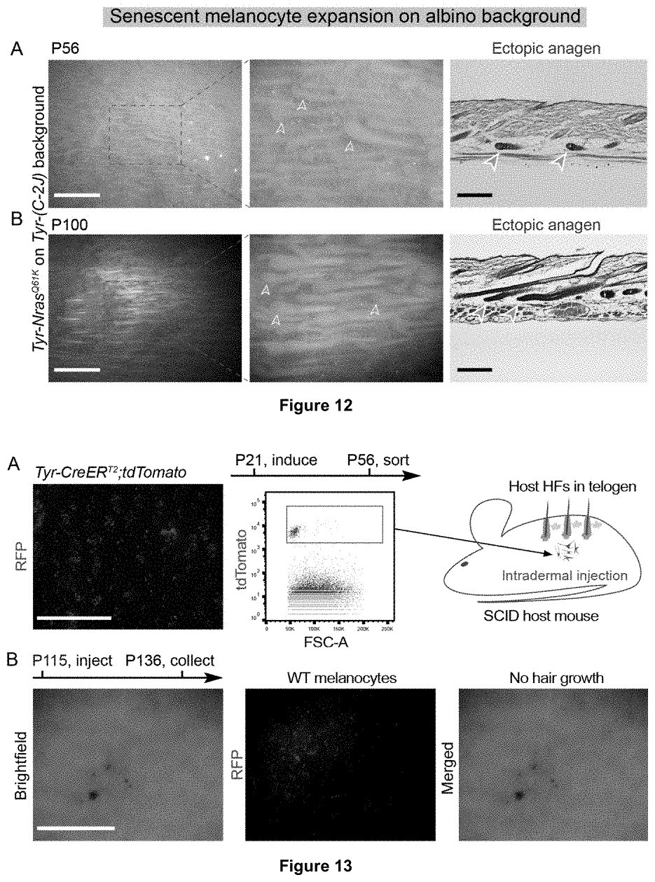

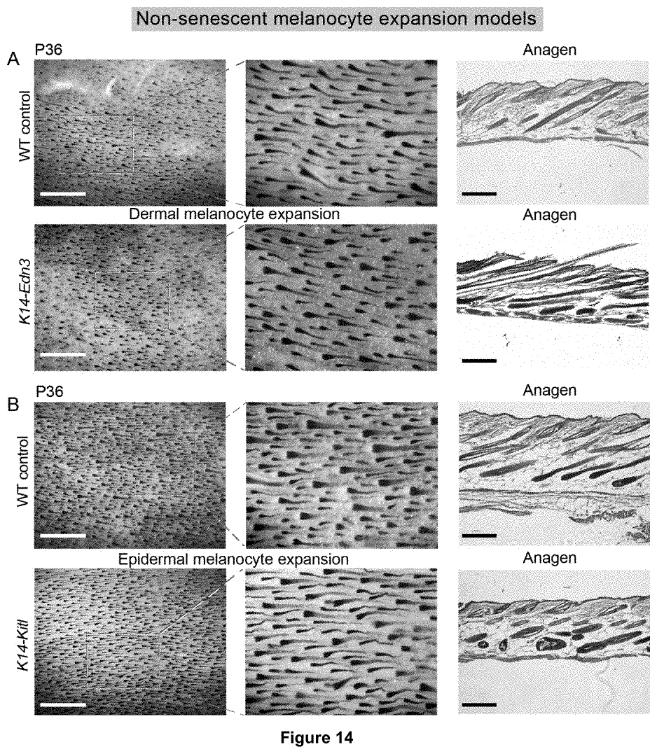

Next, it was considered whether osteopontin plays functional role in hair growth phenotype of Tyr-Nras^{Q61K} mice and if it is sufficient to induce new hair cycle in WT mice. Tyr-Nras^{Q61K};Spp1^{-/-} mice were generated to test if osteopontin deletion rescues hair cycle quiescence in nevus skin. Indeed, compared to Tyr-Nras^{Q61K} mice, whose HFs start cycling ectopically already at P23 (FIGS. 4G and 11), ectopic anagen is first evident in Tyr-Nras^{Q61K};Spp1^{-/-} mice only at P52, in the mid-second telogen (FIGS. 7L and 15D). By examining hair cycle in Tyr-NrasQ^{61K} ;Spp1^{-/-} and control Spp1^{-/-} mice between days P18-69 (n≥2 per time point per genotype), it was established that it progresses largely normally in control mice null for Spp1. These results indicate that osteopontin is dispensable for normal hair cycle progression, but may play a significant role in hair cycle hyper-activation in nevus skin. Also, appearance of ectopic anagen HFs in Tyr-Nras^{Q61K};Spp1^{-/-} skin after P52 (FIG. 15) indicates that other signaling factors, part of SASP (FIGS. 12A-12B) can compensate for osteopontin loss.

Considering that osteopontin becomes prominently upregulated in immune cells and fibroblasts at the edge of healing skin wounds (Liaw et al., 1998; Mori et al., 2008), it was asked if it might mediate wound-induced hair cycle activation phenomenon, when HFs in WT mice enter ectopic anagen at the wound margin. A previous study implicated Tnf as one of the signaling mediators of this phenomenon (Wang et al., 2017b). It is shown here that compared to WT mice, Spp1^{-/-} mice show significantly fewer ectopic anagen HFs at the margin of 7 mm full-thickness wounds 11 days post-wounding (FIG. 7M). Also, ectopic anagen was prominently induced 12 days after intradermal injection of osteopontin-soaked beads in WT mice, as compared to control BSA-soaked beads (FIG. 7N). Collectively, these results confirm that osteopontin is sufficient to induce new hair cycle in WT mice, and that it mediates ectopic hair cycling in at least two skin states with inflammatory component, melanocytic nevus and wound healing.

### Osteopontin Effect is Mediated by Immune Cells and in Some Cases Involves Cd44

Myeloid cells are a source and target for osteopontin signaling in the context of various inflammatory conditions, including in skin (Buback et al., 2009; Giachelli et al., 1998; Liaw et al., 1998; Mori et al., 2008). Considering this and prominent increase in Spp1 levels in Tyr-Nras^{Q61K} myeloid cells on bulk (FIGS. 8D and 8E) and single-cell RNA-seq (FIGS. 8G and 21), it was considered whether myeloid cells mediate nevus skin phenotype. First, significantly more tdTomato⁺ cells were observed in skin of P56 Tyr-Nras^{Q61K};LysM-Cre;tdTomato mutant vs. LysM-Cre;tdTomato control myeloid cell-specific reporter mice (FIGS. 8H and 8H′). Spp1 deletion reduced myeloid infiltration of P56 skin in Tyr-Nras^{Q61K};Spp1^{-/-};LysM-Cre;tdTomato mice as compared to Spp1^{-/-};LysM-Cre;tdTomato control mice (FIGS. 8I and 8F). Intradermal injection of osteopontin to P47 LysM-Cre;tdTomato reporter mice led to a significant increase in myeloid cell infiltration after 24 hours as compared to BSA control (FIGS. 8J and 8J′). P54 Tyr-Nras^{Q61K},LysM-Cre;Rosa-rtTA;tetO-DTA mice were also treated with doxycycline to induce DTA-mediated myeloid cell depletion. Significantly fewer ectopic anagen HFs were observed after six days of doxycycline treatment as compared to doxycycline-treated littermate control animals, lacking either Rosa-rtTA or tetO-DTA construct (FIGS. 8K and 8K′).

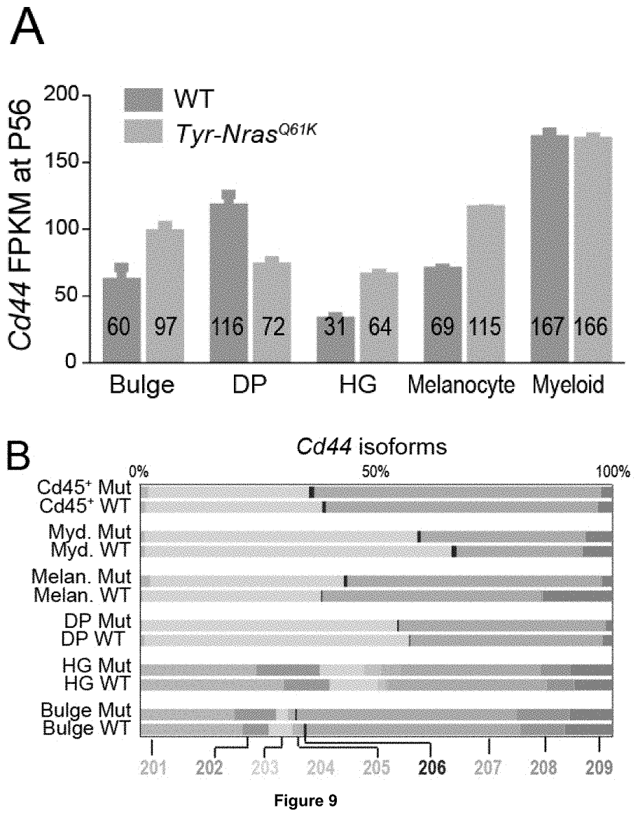

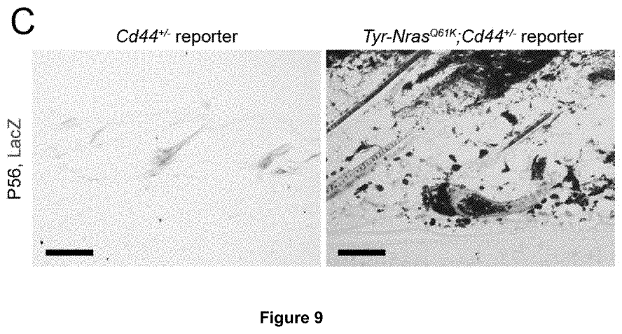

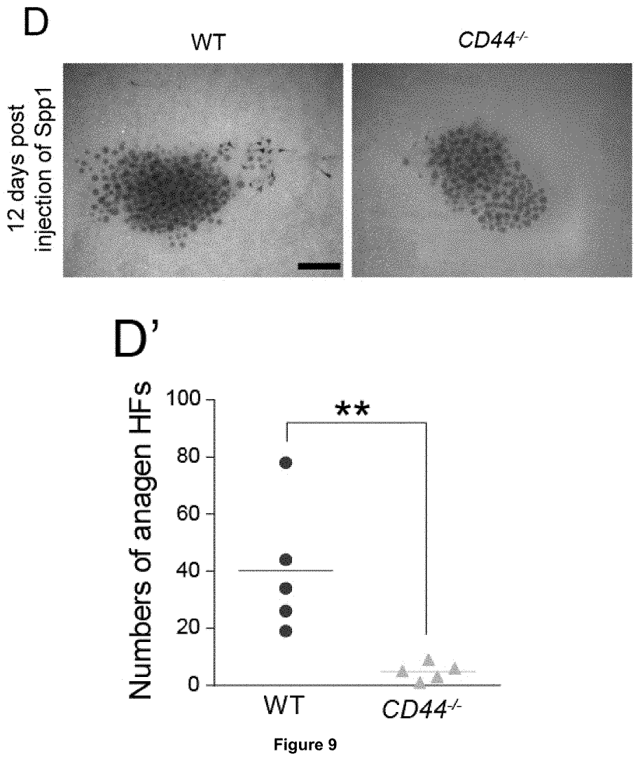

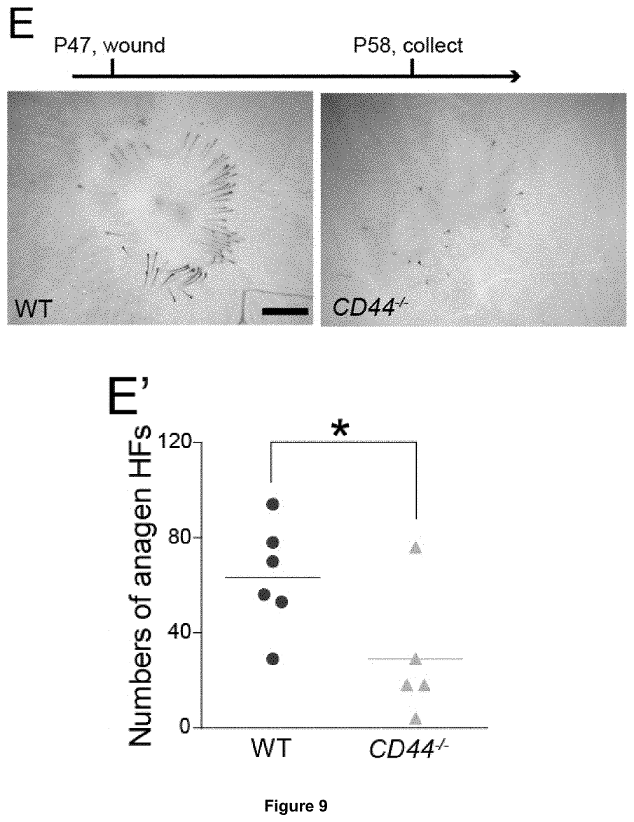

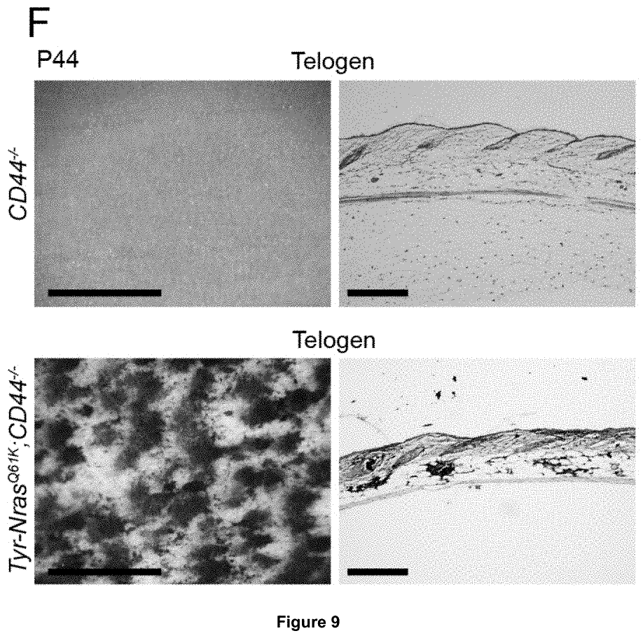

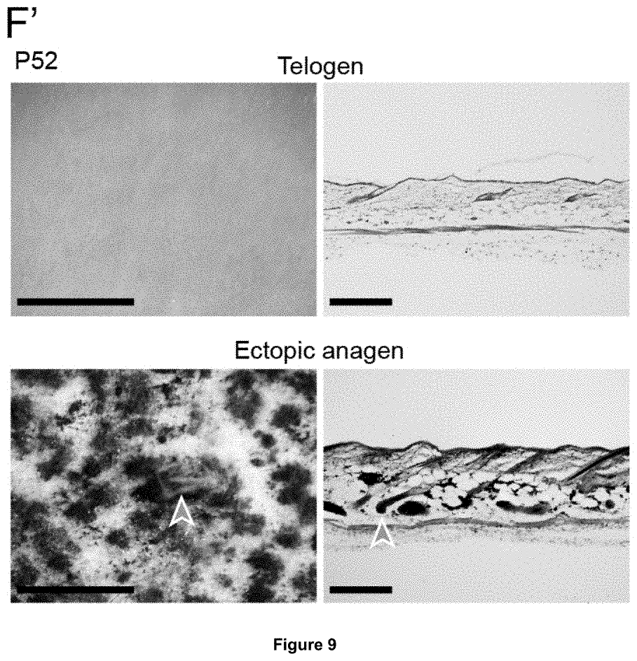

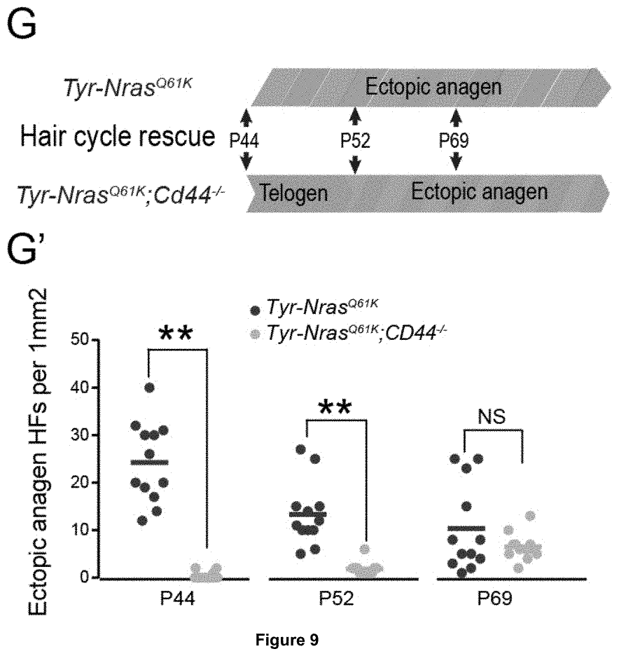

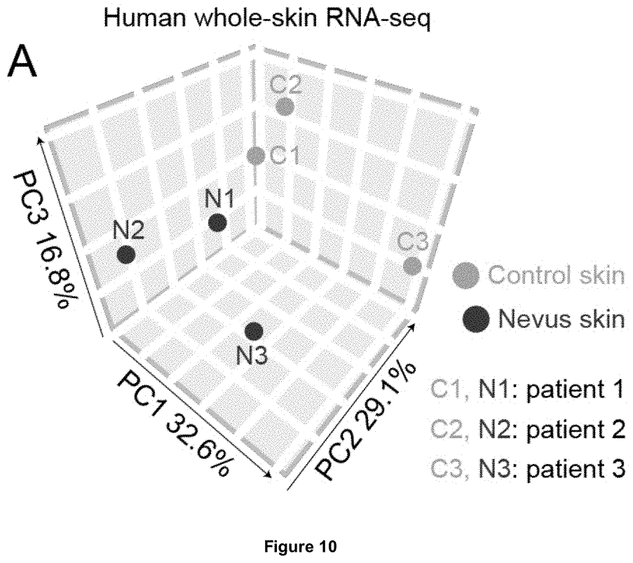

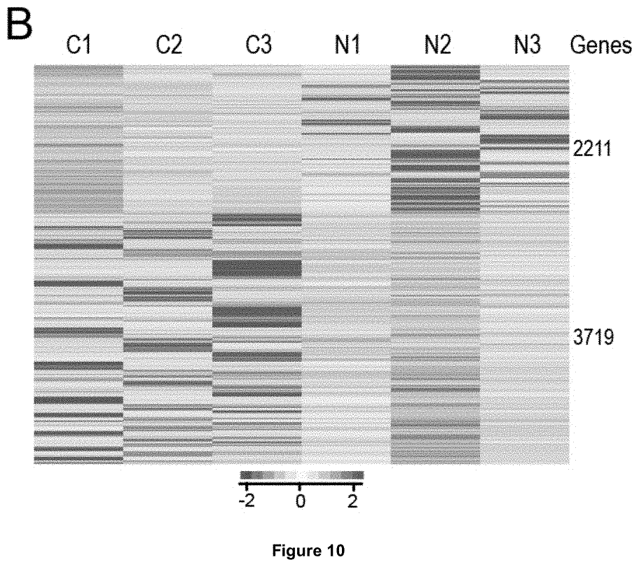

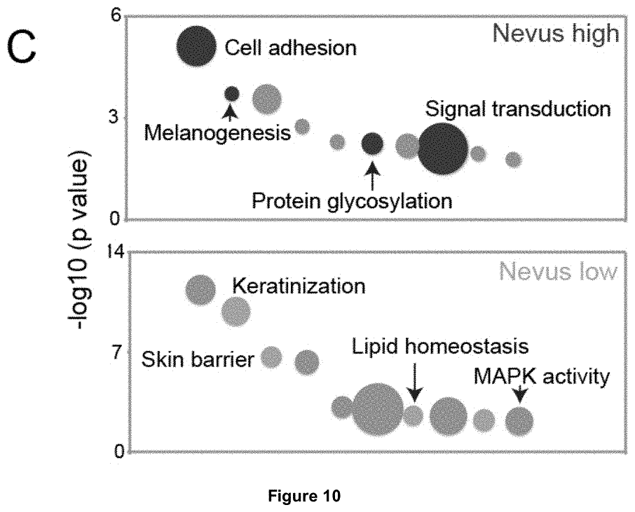

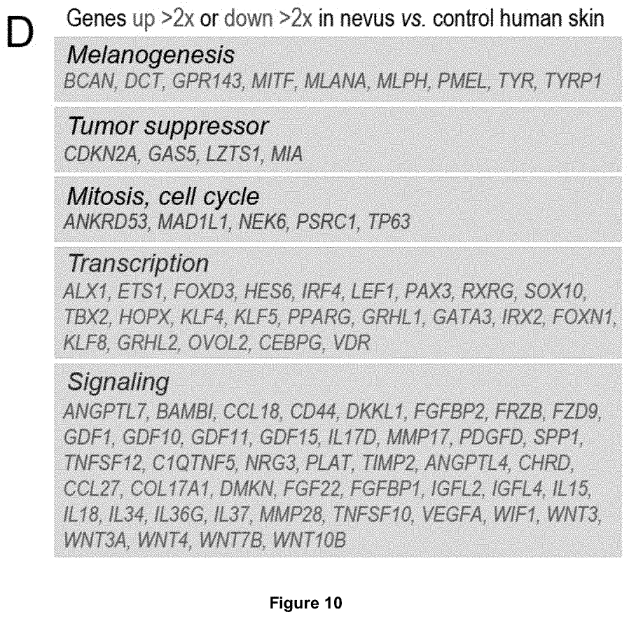

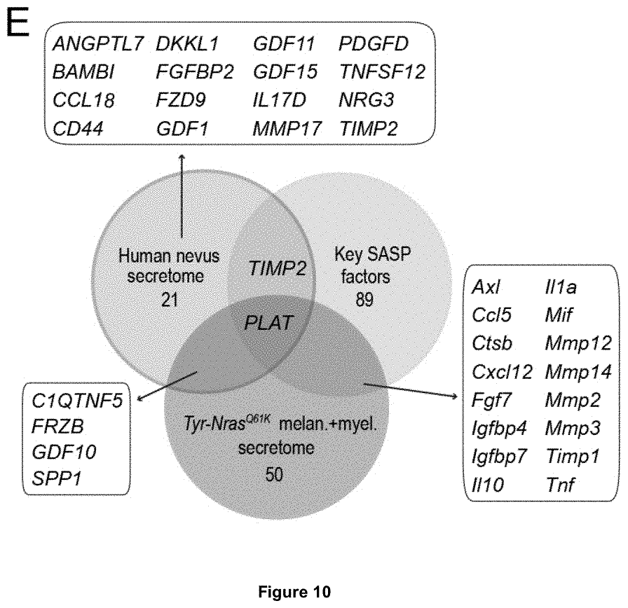

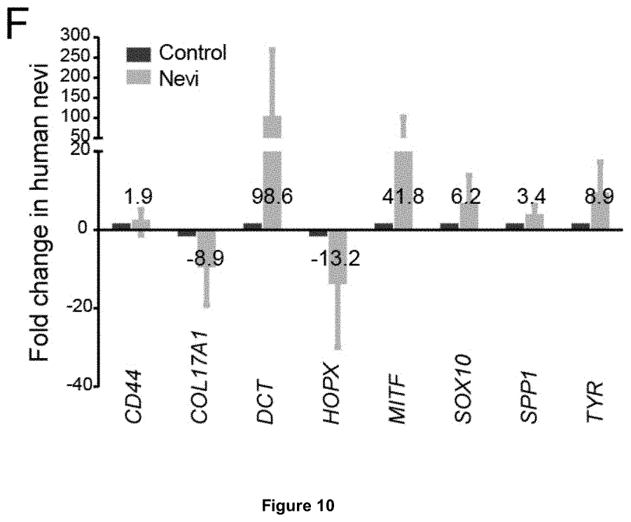

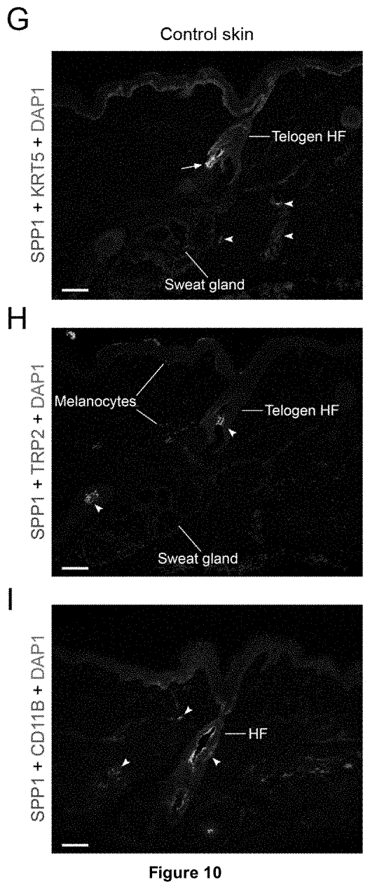

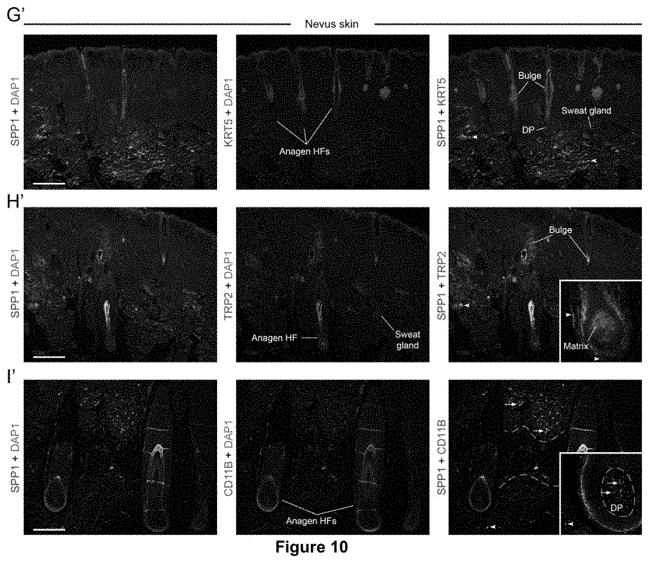

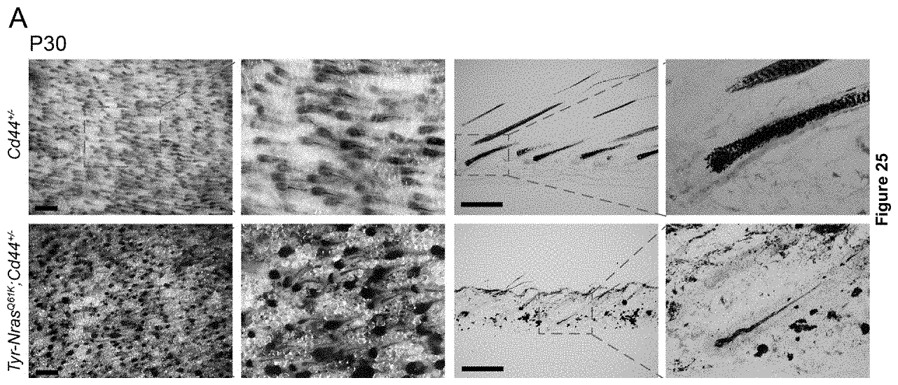

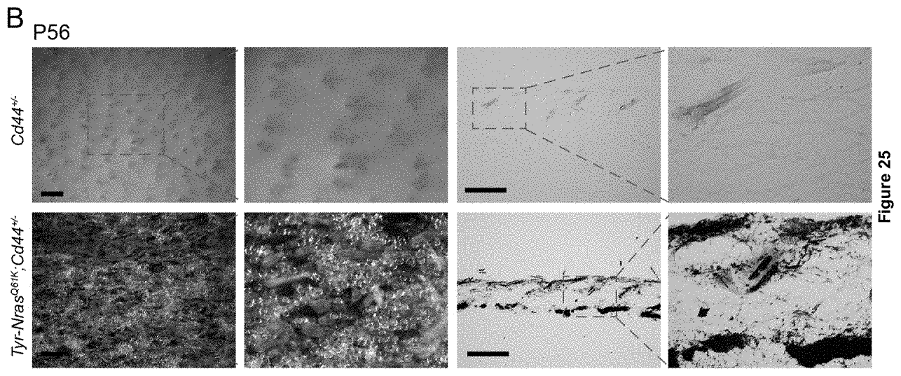

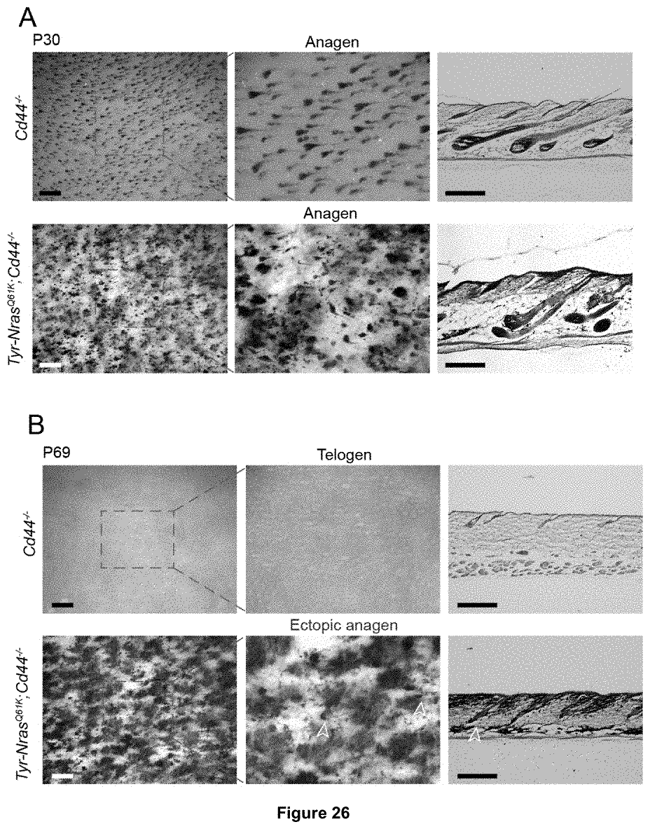

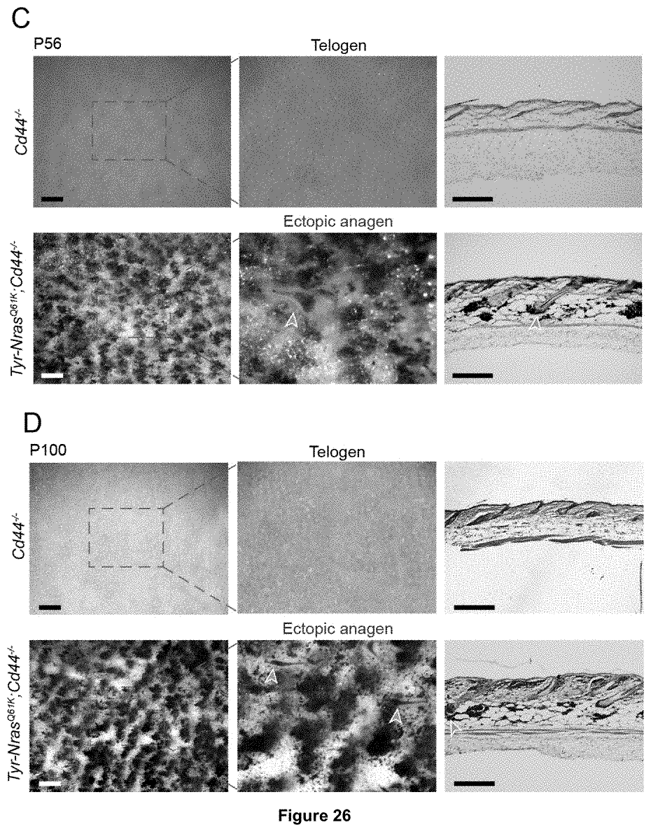

Next, it was examined whether Cd44, a receptor for osteopontin (Weber et al., 1996), mediates its effect in nevus skin. On RNA-seq Cd44 was highly expressed in multiple skin cell types, including bulge and HG progenitors, both in Tyr-Nras^{Q61K} and control mice (FIG. 9A). Cd44 generates alternatively-spliced isoforms, including standard Cd44s and several variable Cd44v isoforms. RNA-seq data for transcript isoforms was profiled and identified cell type-specific Cd44 isoform enrichment patterns (FIG. 9B). Bulge and HG progenitors were particularly enriched for Cd44v isoforms containing exons v6 and v7, that were previously implicated in mediating osteopontin signaling (reviewed in Ponta et al., 2003). Broad expression of Cd44 in the skin was confirmed, including in bulge SCs, on lacZ staining in Tyr-Nras^{Q61K};Cd44^{+/-} and control Cd44^{+/-} reporter mice (FIGS. 9C, 25A and 25B). Functionally, induction of ectopic anagen in response to osteopontin-soaked beads was significantly suppressed in Cd44^{-/-} mutant vs. WT control mice (FIGS. 9D and 9D′). Likewise, significantly fewer ectopic anagen HFs were induced at the wound margin of Cd44^{-/-} mutant vs. WT control mice (FIGS. 9E and 9E′). Cd44 deletion in Tyr-Nras^{Q61K};Cd44^{-/-} mice led to partial rescue of ectopic hair cycling (FIGS. 9F, 9F′, 9G, and 9G′), phenocopying the effect of Spp1 deletion on Tyr-Nras^{Q61K} background. All HFs in Tyr-Nras^{Q61K};Cd44^{-/-} mice were in telogen at P44 (FIG. 9F), and ectopic anagen HFs first appeared at P52, progressively increasing thereafter (n≥2 per time point per genotype) (FIGS. 9G′ and 26A-26D). It was also established that Cd44 deletion alone does not significantly alter normal progression of the first two hair cycles (FIGS. 9F, 9F′ and 26A-26D). These results indicate that hair-growth activating effect of osteopontin in nevus skin involves Cd44 signaling.

### WNT Signaling is Dispensable for Hair Cycle Hyper-Activation in Nevus Skin

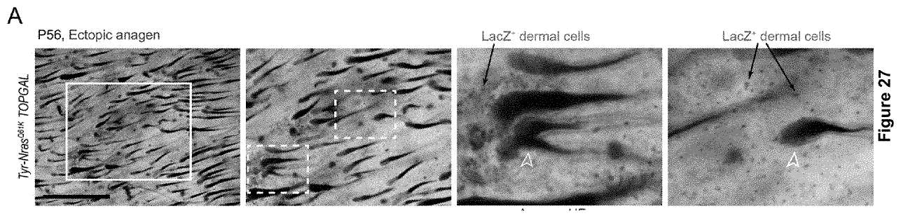

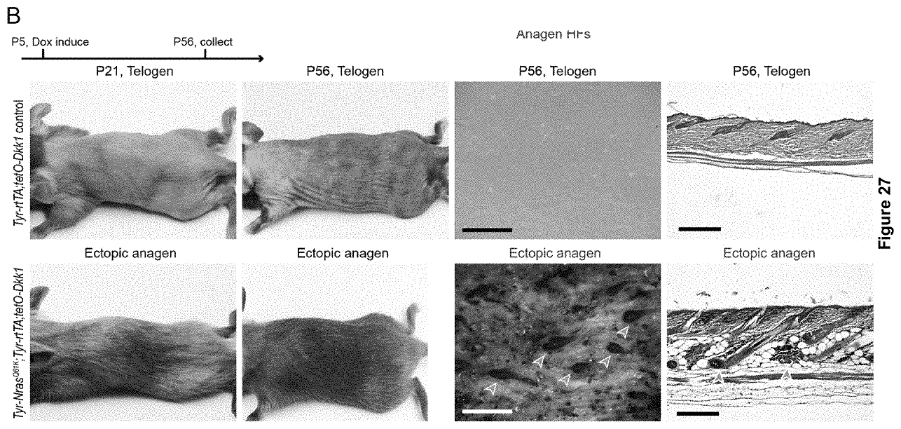

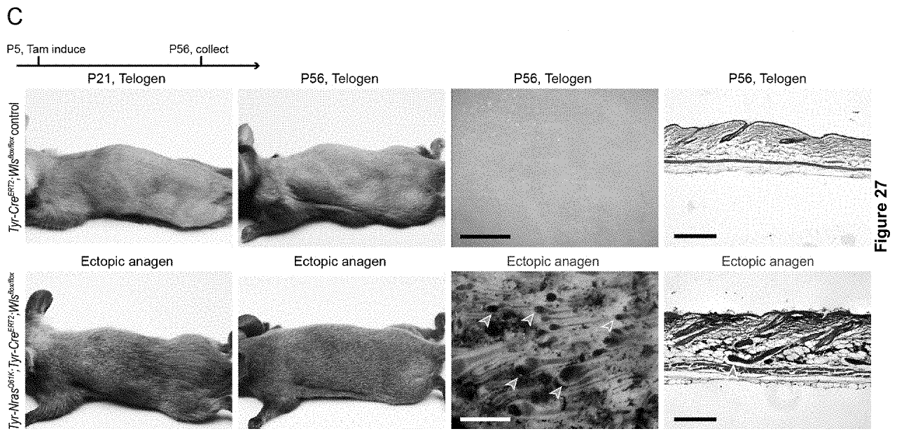

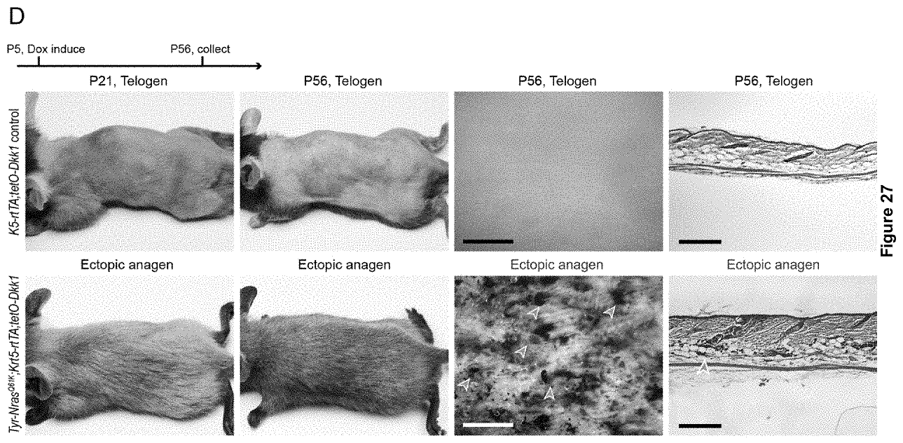

Hyper-activated hair cycling in nevus skin resembles the phenotype of K14-Wnt7a mice that overexpress canonical WNT ligand (Plikus et al., 2011). WNT signaling plays a role in physiological hair cycle activation (Choi et al., 2013; Greco et al., 2009; Kandyba et al., 2013; Lien et al., 2014; Lowry et al., 2005) and it is elevated and drives early stages of melanocytic nevus formation (Pawlikowski et al., 2013). Foci of WNT reporter-active cells were consistently found in the dermis of Tyr-Nras^{Q61L};TOPGAL mice (FIGS. 4F and 28A). At the same time, our RNA-seq analysis suggests that neither melanocytes nor myeloid cells in Tyr-NrasQ^{61K} skin overexpress canonical WNT ligands (FIG. 7D and 8D). To establish functional relevance of WNT signaling in nevus hair cycle phenotype, its activity was blocked using several mouse models. WNT signaling from melanocytes was targeted by either overexpressing soluble WNT antagonist Dkk1 or ablating W1s, necessary for WNT ligand secretion. Both Tyr-Nras^{Q61K};Tyr-rtTA;tetO-Dkk1 mice (induced with doxycycline from P0) and Tyr-Nras^{Q61K};TyrCre^{ERT2};Wls^{flox/flox} mice (induced with tamoxifen between P0-P5) continued to show prominent ectopic anagen at P21 and P56 (FIGS. 27B and 27C). Furthermore, Dkk1 broad overexpression was induced using epithelial-specific promoter in Tyr-NrasQ^{61K};Krt5-rtTA;tetO-Dkk1 mice (induced with doxycycline from P0). However, this also failed to supress ectopic hair cycling (FIG. 27D). At the same time, normal hair cycle activation was prevented in doxycycline-induced Krt5-rtTA,tetO-Dkk1 control mice consistent with the report by Choi et al. (2013). Together, this data indicates that hair cycle hyper-activation in nevus skin does not critically rely on WNT signaling.

### Human Hairy Pigmented Nevi Upregulate Osteopontin Expression

Signaling aspects of hairy pigmented nevi in humans were also examined. Whole-tissue RNA-sect revealed prominent transcriptome differences between hairy nevi and adjacent normal facial skin, as well as patient-to-patient variability (FIGS. 8A-8C). As expected, human nevus skin showed enrichment for melanogenesis genes: BCAN, DCT, GPR143, MITF, MILANA, MLPH, PMEL, TYR and TYRP1. Also, consistent with Tyr-Nras^{Q61K} mouse melanocyte data, human nevi upregulated tumor suppressors CDKN2A, GAS5, LZTS1, MIA and mitosis markers ANKRD53, MAD1L1, NEK6, PSRC1 (FIG. 8D). Among secreted factors, human nevi upregulated several TGFβ/BMP pathway members GDFP1/10/11/15, BAMBI, WNT modulators DKKL1, FRZB, as well as CCL18, IL17D, PDGFD and SPP1 (FIG. 8D). In comparison, only a handful of secretome factors were shared between human hairy nevi and Tyr-Nras^{Q61K} mouse melanocytes and myeloid cells, suggesting species-specific signaling differences (FIG. 8E). Intriguingly, SPP1 was one of such shared factors, which was validated by qRT-PCR (FIG. 8F) and by immunostaining (FIGS. 8G-8I and 8G′-8I′). Increased SPP1 expression was prominent in the interfollicular dermis, were many SPP1-positive cells also co-express macrophage marker CD11B (FIG. 8I′). Also, many SPP1/TRP2 double-positive melanocytes were seen in nevus skin surrounding bulge and bulb regions of anagen HFs (FIG. 8H′). Together, this data suggests that SPP1 overexpression correlates with hair growth over-activation in human nevi.

### Discussion of Example 5 Findings

Genome-wide cross comparison of purified melanocytes, bulge, DP, sHG, and myeloid cells via FACS sorting, was used to identify osteopontin as a new SASP molecule for hair stem cell activation in the senescent melanocytic nevi skin. Osteopontin is expressed in the mouse uterus (Qi et al., 2014), but its function seems redundant because osteopontin knockout (Spp1^{-/-}) mice are viable and normal grossly (Liaw et al., 1998). In the adult, the level of osteopontin expression is upregulated following injury or under other pathological circumstances, such as cell transformation (Mori et al., 2008; Zhou et al., 2005). The expression pattern of osteopontin reflects its multifunctional feature in response to diverse stimuli (Cooper et al., 2005; Liu et al., 2004). In the normal skin, it is absent in the bulge and sHG, suggesting a non-permissive role of osteopontin in the SC compartments. Although it is expressed in melanocytes, DP, and dermal myeloid cells in a hair cycle-dependent manner, osteopontin is dispensable for normal HF SC regeneration, as normal cyclic hair growth was observed in Spp1^{-/-} mice. However, it was involved in precocious hair growth in the nevus skin, evidenced by loss of ability to regenerate HF SC when osteopontin is deleted in Spp1^{-/-} mice. This finding highlights its general role as a stress sensor that was demonstrated previously in connection with altered wound healing in Spp1^{-/-} mice (Liaw et al., 1998).

Skin operates as a complex organ consisting of different structures with multiple cell types. Their interaction with each contributing to specific function is involved in SC regeneration. Osteopontin was upregulated in melanocytes, bulge, DP, and myeloid population in the nevus skin. Normal HF SC regeneration is controlled by both its immediate niche cell DP (Rendl et al., 2008) and other cell types in the skin such as adipocytes (Festa et al., 2011; Plikus et al., 2008). On one hand, upregulation of osteopontin in HF niche cell DP (9.8-fold) and bulge SC (73.9-fold) suggests that the mode of osteopontin action on HF SC regeneration can be direct through modulation of niche and SC themselves. On the other hand, the high osteopontin expressing senescent melanocytes (11.8-fold) can recruit myeloid cells and increase osteopontin expression on those cells (30.2-fold), suggesting a positive feedback loop to sensitize other cell types, and to produce osteopontin in the nevus skin. However, this premature anagen hair phenotype was lost when myeloid cells are depleted, suggesting an important role of myeloid cells in promoting hair growth in the nevus skin. Although SCs are regulated by their niches which are usually nearby and are able to produce rapid-response paracrine factors, extra-niches in the skin which contain of various cell types with distinct functions also participate normal SC regeneration. In this regard, our finding supports the notion of extra-niche cell interaction with SC to regulate hair cycle in the presence of senescent cells.

While the origin of osteopontin is complex (it could be derived from senescent melanocytes or myeloid cells), it appears to have an active role in regeneration of HF SC in senescent skin. A feature of SASP is to attract immune cells. Senescent cells in tumors can recruit immune cells through the SASP and allow tumor clearance (Xue et al., 2007), whereas prolonged SASP can enhance tumor proliferation, migration, and invasion (Bavik et al., 2006), demonstrating distinct functions of the immune cells in the senescent environment. SASP factors are mostly characterized in culture and found in senescent cells (Capell et al., 2016; Copp: et al., 2008; Pawlikowski et al., 2013), they have an altered expression profile enriched in growth factors, chemokines, and ECM remodeling enzymes. The list of nevus-derived factors is extensive. An array of SASP factors (cytokines and chemokines) such as CCL17, CXCL9, CXCL3, and IL1b were found in the myeloid cells but not in the senescent melanocytes, whereas matrix metalloproteinases (MMP3, 12, and 14 in myeloid and MMP11 and 23 in senescent melanocytes, respectively) were altered in both cell types. The expression of osteopontin has an effect on the behavior of neighboring cells in transducing paracrine/autocrine signaling.

### Experimental Procedures Used in Example 5

### Mouse Models

All experiments were performed in accordance with University of California Irvine's Animal Care and Use Committee guidelines. B6.129S6(Cg)-Spp1^{tmlBlh/J} (Lia w et al., 1998), B6.129(Cg)-Cd44^{tmlHbg}/J (Protin et al., 1999), B6(Cg)-Tyr^{c-2J}/J (Townsend et al., 1981), B6.CB17-Prkdc^{scid}/SzJ (Blunt et al., 1995), B6;129S-Sox2^{tm2Hoch}/J (Arnold et al., 2011), Tyr-Nras^{Q61K} (Ackermann et al., 2005) were purchased from The Jackson Laboratory. Tetracycline controlled triple mutant mice of myeloid lineage specific depletion were created by crossing LysM-Cre, Rosa-reTA and TetODTA (Chen et al., 2015).

### EdU Pulse Chase

One-month-old mice were injected with EdU (5 μg/g body weight) via i.p. daily for seven consecutive days, followed by a chase period of 8 weeks. Mouse dorsal skin was harvested; half was fixed in 4%PFA, embedded in paraffin and examined by immunohistochemistry (IHC) using EdU imaging kit (Molecular Probe). The other half was used for flow cytometry quantification using EdU flow kit (Molecular Probe). Both MC showing Edu positive cells among total numbers of follicles and FACS analyzing triple positive CD34⁻CD49f⁺ Edu⁺ cells were used to quantify EdU positive cells. At least two sections per animal and three to five animals per group were used for analysis.

### Intradermal Potein Injections

Intradermal delivery of protein-soaked agarose beads was performed according to Plikus 2008. Briefly, recombinant mouse SPP1 protein (R&D) was reconstituted in 0.1%BSA at a final concentration of 1.3 mg/ml. Affi-gel blue gel beads (Bio-Rad) were washed three times in sterile PBS and then resuspended with recombinant protein (vol/vol) in 0.1%BSA on ice for 1 hr before injection. For both recombinant SPP1 protein and BSA control, four consecutive daily injections, including 24, 48, and 72 hrs after first bead implantation, were performed to the same skin region using a 26G needle to create a pouch, then delivered by a glass micropipette, introducing about 100 beads/20 μl bead solution using a microinjector under the back skin from p51 to p53.

### Histology and Immunohistochernistry

For paraffin-embedded sections, back skins were fixed with 4% (vol/vol) paraformaldehyde (PFA) overnight at 4° C., followed by dehydration with 20%, 50%, and 70% of ethanol. Sections were permeabilized for 15 min in PBS +0.1% Triton X-100 (PBST) and blocked for at least 1 hr at room temperature using PBST +3%BSA. Mouse Abs were blocked with M.O.M. block kit according to manufacturer's instructions. Primary antibodies (Abs) were incubated overnight at 4° C. and secondary Abs were incubated 1 hr at RT. Frozen sections were cryopreserved in Optimal Cutting Temperature compound (OCT). The following antibodies and dilutions were used. Spp1 (1:20, goat, R&D). CD45 (1:100, rabbit, BD Biosciences), F4/80 (1:100, rabbit, BD Biosciences). Nuclei were stained with 4060-diamidino-2-phenylindole (DAPI). For β-gal staining, think sections (20 μm) were incubated in 1 mg/ml X-gal substrate in PBS with 1.3 mM MgCl₂, 3 mM K₃Fe(CN)6, and 3 mM K₄Fe(CN)6 at 37° C. overnight. Hematoxylin and Eosin staining was performed using standard methods. Percent positive area was calculated using ImageJ. All images were captured with a Nikon dissecting or Nikon Ti-E Upright microscope.

### Western Blot

Single cells from mouse whole back skin were lysed in RIPA buffer containing a cocktail of protease inhibitors (Roche). 25 μg of each cell lysates (n=3 samples per group) were loaded onto a 12% separating Bis-Tris gel. The proteins were transferred to a nitrocellulose membrane. The membrane was incubated with the Spp1. primary antibody or anti-GAPDH at a concentration of 2.5 μg ml⁻¹. The blot was developed with Enhanced Chemiluminescence Plus Developer.

### Real-Time PCR

Total RNA from FACS sorted cells was extracted using RNeasy Mini Kit (QIAGEN) coupled with its on-column DNase digestion protocol. This total RNA was then reverse-transcribed by Superscript III (Life Technologies) in the presence of Oligo-dT. The Full length cDNA was normalized to equal amount using house keep genes GAPDIT or 18s.

### Cell Isolation and Sorting

For single cell suspension, the back skin was incubated in Dispase II solution (Roche) to separate epidermis from dermis. Dermis and/or epidermis was digested into single cells with Collagenase I (Life Technologies) at 37° C. These skin single cells were filtered with strainers (70 μM, followed by 40 μM). Viability dye was used to exclude dead cells. Gated live cells were sorted on FACSAria II sorters (BD Biosciences). FACS acquisition was performed on LSRII flow cytometer (BD Biosciences) and then analyzed with FlowJo software (FlowJo).

### Bulk and Single-Cell RNA-Sequencing

Total RNAs from FACS sorted cells including three biological triplicates with RNA integrity number (RIN) >9.1 determined by Agilent 2100 Bioanalyzer Pico chip were selected for cDNA synthesis and amplification. 1 ng of mRNA was used for full length cDNA synthesis, followed by PCR amplification according to Smart-seq2 standard protocol, cDNA libraries were constructed using the Nextera DNA Sample Preparation Kit (Illumina). The libraries were sequenced on the Illumina Next-Seq500 system to an average depth of 10-30 million reads per library using paired 43 bp reads.

For single cell RNA-seq (scRNA-seq) sample preparations on the C1 platform, sorted cells from mouse back skins were captured using the Fluidigm C1 chips according to Fluidigm C1 protocol. A concentration of 200,000-350,000 cells per ml was used for chip loading. After cell capture, chips were examined visually under the microscope to identify the capture rate and empty chambers or chambers with multiple cells were excluded from later analysis. cDNAs were synthesized and amplified on Fluidigm C1 Single-Cell Auto Prep System with Clontech SMARTer Ultra Low RNA kit and ADVANTAGE-2 PCR. kit (Clontech). scRNA-seq libraries were constructed in 96-well plates using the Illumina Nextera XT DNA Sample Preparation kit according to Fluidigm C1 manual. Multiplexed libraries were analyzed on Agilent 2100 Bioanalyzer for fragment distribution and quantified using Kapa Biosystem's universal library quantification kit. Libraries were sequenced as 75 bp paired-end reads on the Illumina Next-Seq500 platform. RNA-seq reads were first aligned using STAR v.2.4.2a (Dobin et al,, 2013) with parameters ‘—outFilterMismatchNmax 10’—outFilterMismatchNoverReadLmax 0.07—outFilterMultimapNmax 10′ to the reference mouse genome (mm10/genocode,vM8) Gene expression level was quantified using RSEM v.1.2.25 (Li and Dewey, 2011) with expression values normalized into Fragments Per Kilobase of transcript per Million mapped reads (FPKM). Samples displaying >9,000,000 uniquely mapped reads and >60% uniquely mapping efficiency were considered for downstream analyses. Differential expression analysis was performed using edgeR v.3.2.2 (Robinson et al., 2010) on protein-coding genes and lncRNAs. Differentially expressed genes were selected by using fold change (FC)≥2, false discovery rate (FDR)<0.05 and counts per million reads (CPM)≥2.

### Example 5 References

Ackermann, J., Frutschi, M., Kaloulis, K., McKee, T., Trumpp, A., and Beermann, F. (2005). Metastasizing melanoma formation caused by expression of activated N-RasQ61K on an INK4a-deficient background. Cancer research 65, 4005-4011.

Ali, N., Zirak, B., Rodriguez, R. S., Pauli, M. L., Truong, H. A., Lai, K., Aim, R., Corbin, K., Lowe, M. M., Scharschmidt, T. C., et al. (2017). Regulatory T Cells in Skin Facilitate Epithelial Stem Cell Differentiation. Cell 169, 1119-1129 e1111.

Amberg, N., Holcmann, M., Stulnig, G., and Sibilia, M. (2016). Effects of Imiquimod on Hair Follicle Stem Cells and Hair Cycle Progression. The Journal of investigative dermatology 136, 2140-2149.

Andriani, G. A., Almeida, V. P., Faggioli, F., Mauro, M., Tsai, W. L., Santambrogio, L., Masiov, A., Gadina, M., Campisi, J., Vijg, J., et al. (2016). Whole Chromosome Instability induces senescence and promotes SASP. Sci Rep 6, 35218.

Aoki, H., Tomita, H., Hara, A., and Kunisada, T. (2015). Conditional Deletion of Kit in Melanocytes: White Spotting Phenotype Is Cell Autonomous. The Journal of investigative dermatology 135, 1829-1838.

Argyris, T. S. (1956). The effect of wounds on adjacent growing or resting hair follicles in mice, AMA Arch Pathol 61, 31-36.

Arnold, K., Sarkar, A., Yram, M. A., Polo, J. M., Bronson, R, Sengupta, S., Seandel, M., Geijsen, N., and Hochedlinger, K. (2011). Sox2(+) adult stem and progenitor cells are important for tissue regeneration and survival of mice. Cell stern cell 9, 317-329.

Barbosa-Souza, V., Contin, D. K., Filho, W. B., de Araujo, A. L., Irazusta, S. P., and da Cruz-Hofling, M. A. (2011). Osteopontin, a chemotactic protein with cytokine-like properties, is up-regulated in muscle injury caused by Bothrops lanceolatus (fer-de-lance) snake venom. Toxicon: official journal of the International Society on Toxinology 58, 398-409.

Bavik, C., Coleman, I., Dean, J. P., Knudsen, B., Plymate, S., and Nelson, P. S. (2006). The gene expression program of prostate fibroblast senescence modulates neoplastic epithelial cell proliferation through paracrine mechanisms. Cancer research 66, 794-802.

Blanpain, C., and Fuchs, E. (2014). Stem cell plasticity. Plasticity of epithelial stem cells in tissue regeneration. Science 344, 1242281.

Blunt, T., Finnie, N. J., Taccioli, G. E., Smith, G. C., Demengeot, j., Gottlieb, T. M., Mizuta, R., Varghese, A. J., Alt, F. W., Jeggo, P. A., et al. (1995). Defective DNA-dependent protein kinase activity is linked to V(D)J recombination and DNA repair defects associated with the murine scid mutation. Cell 80, 813-823.

Buback, F., R,enkl, A. C., Schulz, G., and Weiss, J. M. (2009). Osteopontin and the skin: multiple emerging roles in cutaneous biology and pathology. Exp Dermatol 18, 750-759.

Capell, B. C., Drake, A. M., Zhu, J.. Shah, P. P., Dou, Z., Dorsey, Simola, D. F., Donahue, G, Sammons, M., Rai, T.S., et al. (2016). MLL1 is essential for the senescence-associated secretory phenotype, Genes & development 30, 321-336.

Castellano, D., Paus, R, and Perez-Moreno, M. (2014). Macrophages contribute to the cyclic activation of adult hair follicle stem cells. PLoS biology 12, e1002002.

Chen, C. C., Wang, L, Plikus, M. V., Jiang, T. X., Murray, P. J., Ramos, R., Guerrero-Juarez, C. F., Hughes, M. W., Lee, O. K., Shi, S., et al. (2015). Organ-level quorum sensing directs regeneration in hair stem cell populations. Cell 161, 277-290.

Chen, T., Heller, E., Beronja, S., Oshimori, N., Stokes, N., and Fuchs, E. (2012). An RNA interference screen uncovers a new molecule in stem cell self-renewal and long-term regeneration. Nature 485, 104-108.

Chiche, A., Le Roux, I., von Joest, M., Sakai, H., Aguin. S. B., Cazin, C., Salam, R., Fiette, L., Alegria, O., Flamant, P., et al. (2017), Injury-Induced Senescence Enables In Vivo Reprogramming in Skeletal Muscle. Cell stem cell 20, 407-414 e404.

Choi, Y. S., Zhang, Y., Xu, M., Yang, Y., Ito, M., Peng, T., Cui, Z., Nagy, A., Hadjantonakis, A. K., Lang, R. A., et al. (2013). Distinct functions for Wnt/beta-catenin in hair follicle stem cell proliferation and survival and interfollicular epidermal homeostasis. Cell stem cell 13, 720-733.

Cooper, L., Johnson, C., Bursiem, F., and Martin, P. (2005). Wound healing and inflammation genes revealed by array analysis of ‘macrophageless’ PU.1 null mice. Genome biology 6, R5.

Coppe, J. P., Desprez, P. Y., Krtolica, A., and Campisi, J. (2010). The senescence-associated secretory phenotype: the dark side of tumor suppression. Annu Rev Pathol 5, 99-118.

Coppe, J. P., Patil, C. K., Rodier, F., Sun, Y., Munoz, D. P., Goldstein, J., Nelson, P. S., Desprez, P. Y., and Campisi, J. (2008). Senescence-associated secretory phenotypes reveal cell-nonautonomous functions of oncogenic RAS and the p53 tumor suppressor. PLoS biology 6, 2853-2868.

Cotsarelis, G., Sun, T. T., and Lavker, R. M. (1990). Label-retaining cells reside in the bulge area of pilosebaceous unit: implications for follicular stem cells, hair cycle, and skin carcinogenesis. Cell 61, 1329-1337.

Dankort, D., Curley, D. P., Cartlidge, R. A., Nelson, B., Karnezis, A. N., Damsky, W. E., Jr., You, M. J., DePinho, R. A., McMahon, M., and Bosenberg, M. (2009). Braf(V600E) cooperates with Pten loss to induce metastatic melanoma. Nat Genet 41, 544-552.

Demaria, M., Ohtani., N., Youssef, S. A., Rodier, F., Toussaint, W., Mitchell, J. R., Laberge, R. M., Vijg, J., Van Steeg, H., Dolle, M. E., et al. (2014). An essential role for senescent cells in optimal wound healing through secretion of PDGF-AA. Dev Cell 31, 722-733.

Dhomen, N., Reis-Filho, J. S., da Rocha Dias, S., Hayward, R., Savage, K., Delmas, V., Larue, L., Pritchard, C., and Marais, R., (2009). Oncogenic Braf induces melanocyte senescence and melanoma in mice. Cancer Cell 15, 294-303.

Dikovskaya, D., Cole, J. J., Mason, S. M., Nixon, C., Karim, S. A., McGarry, L., Clark, W., Hewitt, R. N., Sammons, M. A., Zhu, j., et al. (2015). Mitotic Stress Is an Integral Part of the Oncogene-Induced Senescence Program that Promotes Multinucleation and Cell Cycle Arrest. Cell Rep 12, 1483-1496.

Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.

Donati, G., Proserpio, V., Lichtenberger, B. M., Natsuga, K., Sinclair, R., Fujiwara, H., and Watt, F. M. (2014), Epidermal Wnt/beta-catenin signaling regulates adipocyte differentiation via secretion of adipogenic factors. Proceedings of the National Academy of Sciences of the United States of America 111, E1501-1509.

Driskell, R. R., Giangreco, A., Jensen, K. B., Mulder, K. W., and Watt, F. M. (2009). Sox2-positive dermal papilla cells specify hair follicle type in mammalian epidermis. Development 136, 2815-2823,

Eichberger, T., Regl, G., Ikram, M. S., Neill, G. W., Philpott, M. P., Aberger, F., and Frischauf, A. M. (2004). FOXE1, a new transcriptional target of GLI2 is expressed in human epidermis and basal cell carcinoma. The Journal of investigative dermatology 122, 1180-1187.

Festa, E., Fretz, J., Berry, R., Schmidt, B., Rodeheffer, M., Horowitz, M., and Horsley, V. (2011). Adipocyte lineage cells contribute to the skin stem cell niche to drive hair cycling. Cell 146, 761-771.

Folgueras, Guo. X., Pasolli, H. A., Stokes, N., Polak, L., Zheng, D.. and Fuchs, E. (2013). Architectural niche organization by LHX2 is linked to hair follicle stem cell function. Cell stem cell 13, 314-327.

Freund, A., Orjalo, A. Desprez, P. Y., and Campisi, J. (2010), Inflammatory networks during cellular senescence: causes and consequences. Trends Mol Med 16, 238-246.

Gay, D., Kwon, O., Zhang, Z,, Spata, M., Plikus, M. V., Holler, P. D., Ito, M., Yang, Z., Treffeisen, E., Kim, C. D., et al. (2013). Fgf9 from dermal gammadelta T cells induces hair follicle neogenesis after wounding. Nat Med 19, 916-923.

Giachelli, C. M., Lombardi, D., Johnson, R. J., Murry, C. E., and Almeida. M. (1998). Evidence for a role of osteopontin in macrophage infiltration in response to pathological stimuli in vivo. Am J Pathol 152, 353-358.

Greco, V., Chen, T., Rendl, M., Schober, M., Pasolli, H. A., Stokes, N., Dela Cruz-Racelis, J., and Fuchs, E. (2009). A two-step mechanism for stem cell activation during hair regeneration. Cell stem cell 4, 155-169.

Herranz, N., Gallage, S., Mellone, M., Wuestefeld, T., Klotz, S., Hanley, C. J., Raguz, S., Acosta, J. C., Innes, A. J., Banito, A., et al. (2015). mTOR regulates MAPKAPK2 translation to control the senescence-associated secretory phenotype. Nature cell biology 17, 1205-1217.

Horsley, V., Aliprantis, A. O., Polak, L., Glimcher, L. H., and Fuchs, E. (2008). NFATc1 balances quiescence and proliferation of skin stem cells. Cell 132, 299-310.

Hsu, Y. C., and Fuchs, E. (2012). A family business: stem cell progeny join the niche to regulate homeostasis. Nat Rev Mol Cell Biol 13, 103-114.

Hsu, Y. C., Li, L., and Fuchs, E. (2014). Emerging interactions between skin stem cells and their niches. Nat Med 20, 847-856.

Hsu, Y. C., Pasolli, H. A., and Fuchs, E. (2011). Dynamics between stem cells, niche, and progeny in the hair follicle. Cell 144, 92-105.

Jaks, V., Barker, N., Kasper, M., van Es, J. H., Snippert, Clevers, H., and Toftgard, R. (2008). Lgr5 marks cycling, yet long-lived, hair follicle stem cells. Nat Genet 40, 1291-1299.

Kandyba, E., Leung, Y., Chen, Y. B., Widelitz, R., Chuong, C. M., and Kobielak, K. (2013). Competitive balance of intrabulge BMP/Wnt signaling reveals a robust gene network ruling stern cell homeostasis and cyclic activation. Proceedings of the National Academy of Sciences of the United States of America 110, 1351-1356.

Kimura-Ueki, M., Oda, Y., Oki, J., Komi-Kuramochi, A., Honda, E., Asada, M., Suzuki, M., and Imamura, T. (2012). Hair cycle resting phase is regulated by cyclic epithelial FGF18 signaling. The Journal of investigative dermatology 132, 1338-1345.

Kretzschmar, K., and Watt, F. M. (2014). Markers of epidermal stem cell subpopulations in adult mammalian skin. Cold Spring Harb Perspect Med 4.

Kunisada, T., Yoshida, H., Yamazaki, H., Miyamoto, A., Hemmi, H., Nishimura, E., Shultz, L. D., Nishikawa, S., and Hayashi, S. (1998). Transgene expression of steel factor in the basal layer of epidermis promotes survival, proliferation, differentiation and migration of melanocyte precursors. Development 125, 2915-2923,

Laberge, R. M., Sun, Y., Orjalo, A. V., Patil, C. K., Freund, A., Zhou, L., Curran, S. C., Davalos, A. R., Wilson-Edell, K. A., Liu, S., et al. (2015). MTOR regulates the pro-tumorigenic senescence-associated secretory phenotype by promoting IL1A translation. Nature cell biology 17, 1049-1061.

Lay, K., Kume, T., and Fuchs, E. (2016). FOXC1 maintains the hair follicle stem cell niche and governs stem cell quiescence to preserve long-term tissue-regenerating potential. Proceedings of the National Academy of Sciences of the United States of America 113, E4506-1515.

Lee, P., Gund, R., Dutta, A. Pincha, N., Rana, I., Ghosh, S., Witherden, D., Kandyba, E., MacLeod, A., Kobielak, K., et al. (2017). Stimulation of hair follicle stern cell proliferation through an IL-1 dependent activation of gammadeltaT-cells. Elife 6.

Li, B., and Dewey, C. N. (2011). RSEM: accurate transcript quantification from RNA-Seq data with or without a reference genome. BMC bioinformatics 12, 323.

Liaw, L., Birk, D. E., Ballas, C. B., Whitsitt, J. S., Davidson, J. M., and Hogan, B. L. (1998). Altered wound healing in mice lacking a functional osteopontin gene (spp1). The Journal of clinical investigation 101, 1468-1478.

Lien, W. H., Polak, L., Lin, M., Lay, K., Zheng, D., and Fuchs, E. (2014). In vivo transcriptional governance of hair follicle stem cells by canonical Wnt regulators. Nature cell biology 16, 179-190.

Lim, X., Tan, S. H., Yu, K. L., Lim, S. B., and Nusse, R. (2016). Axin2 marks quiescent hair follicle bulge stem cells that are maintained by autocrine Wnt/beta-catenin signaling. Proceedings of the National Academy of Sciences of the United States of America 113, E1498-1505.

Liu, Y. N., Karig, B. B., and Chen, J. H. (2004). Transcriptional regulation of human osteopontin promoter by C/EBPalpha and AML-1 in metastatic cancer cells. Oncogene 23, 278-288.

Lowry, W. E., Blanpain, C., Nowak, J. A., Guasch, G, Lewis, L., and Fuchs, E. (2005). Defining the impact of beta-catenin/Tcf transactivation on epithelial stem cells. Genes & development 19, 1596-1611.

Matsumura, H., Mohri, Y., Binh, N. T., Morinaga, H., Fukuda, M., Ito, M., Kurata, S., Hoeijmakers, J., and Nishimura, E. K. (2016). Hair follicle aging is driven by transepidermal elimination of stem cells via COL17A1 proteolysis. Science 351, aad4395.

Michalogiou, C., Vredeveld, L. C,, Soengas, Denoyelle, C., Kuilman, T., van der Horst, C. M., Majoor, D. M., Shay, J. W., Mooi, W. J., and Peeper, D. S. (2005). BRAFE600-associated senescence-like cell cycle arrest of human naevi. Nature 436, 720-724.

Morgan, B. A. (2014). The dermal papilla: an instructive niche for epithelial stem and progenitor cells in development and regeneration of the hair follicle. Cold Spring Harb Perspect Med 4, a015180.

Mori, R., Shaw, T. J., and Martin, P. (2008). Molecular mechanisms linking wound inflammation and fibrosis: knockdown of osteopontin leads to rapid repair and reduced scarring. The Journal of experimental medicine 205, 43-51.

Mosteiro, L., Pantoja, C., Alcazar, N., Marion, R. M., Chondronasiou, D., Rovira, M., Fernandez-Marcos, P. J., Munoz-Martin, M., Blanco-Aparicio, C., Pastor, J., et al. (2016). Tissue damage and senescence provide critical signals for cellular reprogramming in vivo. Science 354.

Muller-Rover, S., Handjiski, B., van der Veen, C., Eichmuller, S., Foitzik, K., McKay, I. A., Stenn, K. S., and Paus, R. (2001). A comprehensive guide for the accurate classification of murine hair follicles in distinct hair cycle stages. The Journal of investigative dermatology 117, 3-15.

O'Regan, A., and Berman, J. S. (2000). Osteopontin: a key cytokine in cell-mediated and granulomatous inflammation. International journal of experimental pathology 81, 373-390.

Osaka, N., Takahashi, T., Murakami, S., Matsuzawa, A., Noguchi, T., Fujiwara, T., Aburatani, H., Moriyama, K., Takeda, K., and Ichijo, H. (2007). ASK1-dependent recruitment and activation of macrophages induce hair growth in skin wounds. J Cell Biol 176, 903-909.

Pawlikowski, J. S., McBryan, T., van Tuyn, J., Drotar, M. E., Hewitt, R. N., Maier, A. B., King, A., Blyth, K., Wu, H., and Adams, P. D. (2013). Wnt signaling potentiates nevogenesis. Proceedings of the National Academy of Sciences of the United States of America 110, 16009-16014.

Plikus, M. V., Baker, R. E., Chen, C. C., Fare, C., de la Cruz, D., And1, T., Maini, P. K., Millar, S. E., Widelitz, R., and Chuong, C. M. (2011). Self-organizing and stochastic behaviors during the regeneration of hair stem cells. Science 332, 586-589.

Plikus, M. V., and Chuong, C. M. (2014). Macroenvironmental regulation of hair cycling and collective regenerative behavior. Cold Spring Harb Perspect Med 4, a015198.

Plikus, M. V., Mayer, J. A., de la Cruz, D., Baker, R. E., Maini, P. K., Maxson, R., and Chuong, C. M. (2008). Cyclic dermal BMP signalling regulates stem cell activation during hair regeneration. Nature 451, 340-344.

Ponta, H., Sherman. L., and Herrlich, P. A. (2003). CD44: from adhesion molecules to signalling regulators. Nat Rev Mol Cell Biol 4, 33-45.

Protin, U., Schweighoffer, T., Jochutn, W., and Hilberg, F. (1999). CD44-deficient mice develop normally with changes in subpopulations and recirculation of lymphocyte subsets. J Immunol 163, 4917-4923.

Qi, Q. R., Xie, Q. Z., Liu, X. L., and Zhou, Y. (2014). Osteopontin is expressed in the mouse uterus during early pregnancy and promotes mouse blastocyst attachment and invasion in vitro. PloS one 9, e104955.

Rendl, M., Polak, L., and Fuchs, E. (2008). BMP signaling in dermal papilla cells is required for their hair follicle-inductive properties. Genes & development 22, 543-557.

Ritschka, B., Storer, M., Mas, A., Heinzmann, F., Ortells, M. C., Morton, J. P., Sansom, Zender, L, and Keyes, W. M. (2017). The senescence-associated secretory phenotype induces cellular plasticity and tissue regeneration. Genes & development 31, 172-183.

Robinson, M. D., McCarthy, D. J., and Smyth, G. K. (2010). edgeR.: a Bioconductor package for differential expression analysis of digital gene expression data. Bioinformatics 26, 139-140.

Roh, M. R., Eliades, P., Gupta, S., and Tsao, H. (2015). Genetics of melanocytic nevi. Pigment Cell Melanoma Res 28, 661-672.

Ruhland, M. K., Loza. A. J., Capietto, A. H., Luo, X., Knolhoff, B. L., Flanagan, K. C., Belt, B. A., Alspach, E., Leahy, K., Luo, J., et al. (2016). Stromal senescence establishes an immunosuppressive microenvironment that drives tumorigenesis. Nat Commun 7, 11762.

Scadden, D. T. (2014). Nice neighborhood: emerging concepts of the stem cell niche. Cell 157, 41-50.

Sennett, R., and Rendl, M. (2012). Mesenchymal-epithelial interactions during hair follicle morphogenesis and cycling. Semin Cell Dev Biol 23, 917-927.

Storer, M., Mas, A., Robert-Moreno, A., Pecoraro, M., Ortelis, M. C., Di Giacomo, V., Yosef, R., Pilpel, N., Krizhanovsky, V., Sharpe, J., et al. (2013). Senescence is a developmental mechanism that contributes to embryonic growth and patterning. Cell 155, 1119-1130.

Takeda, N., Jain, R., Leboeuf, M. R , Padmanabhank, Wang, Q., Li, L., Lu, M. M., Millar, S .E., and. Epstein, J. A. (2013). Hopx expression defines a subset of multipotent hair follicle stem cells and a progenitor population primed to give rise to K6+ niche cells. Development 140, 1655-1664,

Townsend, D., Witkop, C. J., Jr., and Mattson, J. (1981). Tyrosinase subcellular distribution and kinetic parameters in wild type and C-locus mutant C57BL16J mice. The Journal of experimental zoology 216, 113-119.

Vidal, V. P., Chaboissier, M. C., Lutzkendorf, S., Cotsarelis, G, Mill, P., Hui, C. C., Ortonne, N., Ortonne, J. P., and Schedlk (2005). Sox9 is essential for outer root sheath differentiation and the formation of the hair stem cell compartment. Curr Biol 15, 1340-1351.

Wang, L., Siegenthaler, J. A., Dowell, R. D., and Yi, R. (2016), Foxc1 reinforces quiescence in self-renewing hair follicle stem cells. Science 351, 613-617.

Wang, Q., Oh, J. W., Lee, H. L., Dhar, A., Peng, T., Ramos, R., Guerrero-Juarez, C. F., Wang, X., Zhao, R., Cao, X., et al. (2017a). A multi-scale model for hair follicles reveals heterogeneous domains driving rapid spatiotemporal hair growth patterning. Elife 6.

Wang, X.. Chen, H., Tian, R., Zhang, Y., Drutskaya, M. S., Wang, C., Ge, J., Fan, Z., Kong, D., Wang, X., et al. (2017b). Macrophages induce AKT/beta-catenin-dependent Lgr5(+) stem cell activation and hair follicle regeneration through TNF. Nat Commun 8, 14091.

Weber, G. F., Ashkar, S., Glimcher, M. J., and Cantor, H. (1996). Receptor-ligand interaction between CD44 and osteopontin (Eta-1). Science 271, 509-512.

Xin, T., Greco, V., and Mvung, P. (2016). Hardwiring Stem Cell Communication through Tissue Structure. Cell 164, 1212-1225.

Xue, W., Zender, L, Miething, C., Dickins, R. A., Hernando, E., Krizhanovsky, V., Cordon-Cardo, C., and Lowe, S. W. (2007). Senescence and tumour clearance is triggered by p53 restoration in murine liver carcinomas. Nature 445, 656-660.

Yi, R. (2017). Mechanisms of Quiescent Hair Follicle Stem Cell Regulation, Stem Cells.

Yoshimoto, S., Loo, T. M., Atarashi, K., Kanda, H., Sato, S., Oyadomari, S., Iwakura, Y., Oshima, K., Morita, H., Hattori, M., et al. (2013). Obesity-induced gut microbial metabolite promotes liver cancer through senescence secretome. Nature 499, 97-101.

Zhang, B., Tsai, P. C., Gonzalez-Celeiro, M., Chung, O., Boumard, B., Perdigoto, C. N., Ezhkova, E., and Hsu, Y. C. (2016). Hair follicles' transit-amplifying cells govern concurrent dermal adipocyte production through Sonic Hedgehog. Genes & development 30, 2325-2338.

Zhou, Y. W., Dai, D. L., Martinka, M., Su, M., Zhang, Y., Campos, E. I., Dorocicz, I., Tang, L. R., Huntsman, D., Nelson, C., et al. (2005). Osteopontin expression correlates with melanoma invasion. Journal of Investigative Dermatology 124, 1044-1052.

It is contemplated that various combinations or sub-combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to fbrm varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner however, they can also include any third-party instruction of those actions, either expressly or by implication. For example, actions such as “transplanting at least one senescent cell type” includes “instructing the transplanting of at least one senescent cell type” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “at least one factor...” includes “one factor.”

1. A method for enhancing or inducing hair growth in a subject at an area affected by hair loss, comprising:
delivering at least one senescence associated secretory phenotype (SASP) factor, or at least one senescent cell that secretes said at least one SASP factor, to the subject at the area affected by hair loss,
wherein the at least one senescent cell comprises at least one cell that is non-replicative or exhibits a non-replicative phenotype,
wherein the delivery of the at least one senescent cells or of the at least one SASP factor induces one or more of lengthening an anagen phase and shortening a telogen phase of a hair follicle in the area affected by hair loss, and
wherein the lengthening of the anagen phase and/or shortening of the telogen phase of the hair follicle enhances or induces hair growth in the subject at the area affected by hair loss.

2.-63. (canceled)
